SEQUENCE LISTING (27 sequences)

SEQ ID NO: 1 - protein, 3 aa, Artificial Sequence
  SYNTHESIZED
RGD

SEQ ID NO: 2 - protein, 3 aa, Artificial Sequence
  SYNTHESIZED
RDG

SEQ ID NO: 3 - protein, 6 aa, Artificial Sequence
  SYNTHESIZED
  MISC_FEATURE (3)..(3): Xaa = hydroxyproline
GFXGER

SEQ ID NO: 4 - protein, 5 aa, Artificial Sequence
  SYNTHESIZED
YIGSR

SEQ ID NO: 5 - protein, 5 aa, Artificial Sequence
  SYNTHESIZED
IKVAV

SEQ ID NO: 6 - protein, 4 aa, Artificial Sequence
  SYNTHESIZED
REDV

SEQ ID NO: 7 - protein, 4 aa, Artificial Sequence
  SYNTHESIZED
DGEA

SEQ ID NO: 8 - protein, 6 aa, Artificial Sequence
  SYNTHESIZED
VGVAPG

SEQ ID NO: 9 - protein, 5 aa, Artificial Sequence
  SYNTHESIZED
GRGDS

SEQ ID NO: 10 - protein, 3 aa, Artificial Sequence
  SYNTHESIZED
LDV

SEQ ID NO: 11 - protein, 4 aa, Artificial Sequence
  SYNTHESIZED
RGDV

SEQ ID NO: 12 - protein, 5 aa, Artificial Sequence
  SYNTHESIZED
PDSGR

SEQ ID NO: 13 - protein, 7 aa, Artificial Sequence
  SYNTHESIZED
RYVVLPR

SEQ ID NO: 14 - protein, 6 aa, Artificial Sequence
  SYNTHESIZED
LGTIPG

SEQ ID NO: 15 - protein, 3 aa, Artificial Sequence
  SYNTHESIZED
LAG

SEQ ID NO: 16 - protein, 4 aa, Artificial Sequence
  SYNTHESIZED
RGDS

SEQ ID NO: 17 - protein, 4 aa, Artificial Sequence
  SYNTHESIZED
RGDF

SEQ ID NO: 18 - protein, 12 aa, Artificial Sequence
  SYNTHESIZED
HHLGGALQAGDV

SEQ ID NO: 19 - protein, 4 aa, Artificial Sequence
  SYNTHEISZED
VTCG

SEQ ID NO: 20 - protein, 4 aa, Artificial Sequence
  SYNTHESIZED
SDGD

SEQ ID NO: 21 - protein, 6 aa, Artificial Sequence
  SYNTHESIZED
GREDVY

SEQ ID NO: 22 - protein, 5 aa, Artificial Sequence
  SYNTHESIZED
GRGDY

SEQ ID NO: 23 - protein, 6 aa, Artificial Sequence
  SYNTHESIZED
GRGDSP

SEQ ID NO: 24 - protein, 4 aa, Artificial Sequence
  SYNTHESIZED
VAPG

SEQ ID NO: 25 - protein, 9 aa, Artificial Sequence
  SYNTHESIZED
GGGGRGDSP

SEQ ID NO: 26 - protein, 8 aa, Artificial Sequence
  SYNTHESIZED
GGGGRGDY

SEQ ID NO: 27 - protein, 6 aa, Artificial Sequence
  SYNTHESIZED
FTLCFD

(19) United States
(12) Patent Application Publication
(10) Pub. No.: US 2023/0272334 A1
(43) Pub. Date: Aug. 31, 2023

(54) ARTIFICIAL PLACENTA VACCINE FOR ORGAN TRANSPLANTATION

(71) Applicant: Arizona Board of Regents on Behalf of Arizona State University, Tempe, AZ (US)

(72) Inventor: Jessica D. Weaver, Chandler, AZ (US)

(21) Appl. No.: 18/018,220

(22) PCT Filed: Jul. 27, 2021
(86) PCT No.: PCT/US21/70992

§ 371 Date: Jan. 26, 2023

Related U.S. Application Data
(60) Provisional application No. 63/056,975, filed on Jul. 27, 2020.
(60) Provisional application No. 63/056,979, filed on Jul. 27, 2020.

(51) Int. Cl. C12N 5/00; C12N 5/073; A61K 35/54

(52) U.S. Cl. CPC C12N 5/0012; C12N 5/0605; A61K 35/54; C12N 2513/00

(57) ABSTRACT

Devices, and methods for preventing immune rejections are disclosed, in which trophoblasts or trophoblast-like cells are used to induce tolerance toward allogeneic cell and tissue grafts. The devices can be used as artificial placenta vaccines to avoid immunosuppression in organ transplantation.

### CROSS-REFERENCE TO RELATED APPLICATIONS

This application claims priority from U.S. Provisional Application No. 63/056,979 filed Jul. 27, 2021, and U.S. Provisional Application No. 63/056,975 filed Jul. 27, 2021, the entire contents of both of which are hereby incorporated by reference.

### FIELD OF THE INVENTION

The present disclosure relates to organ and cell transplantation devices and methods to avoid transplant rejection.

### BACKGROUND OF THE INVENTION

The ultimate goal in clinical transplantation is robust and specific host immunological tolerance toward transplanted allogeneic tissue and cells. While systemic immunosuppression regimens have saved lives by enabling long-term allogeneic graft survival, they come with a host of potentially severe side effects, including infections and malignancies. Many groups have turned toward local delivery or presentation of immunomodulatory factors, which can eliminate systemic and off-target effects, but have limited opportunity for renewal when exhausted, resulting in a transient effect. One approach to achieve more persistent localized immunomodulation includes the use of tolerogenic cells, such as T regulatory cells (Treg), tolerized mesenchymal stem cells (MSC) or tolerized antigen presenting cells (APC), such as dendritic cells (DC). However, while these cells can be influenced to take on a tolerogenic phenotype in vitro, it is unclear whether, or for how long, these manipulated cells maintain this phenotype in the in vivo environment, and for how long they persist at the site of interest.

With these observations in mind, among others, various aspects of the present disclosure were conceived and developed.

### SUMMARY OF THE INVENTION

In one aspect, the present disclosure provides a placenta-mimicking device comprising (a) allogeneic cells, (b) trophoblasts, and (c) one or more microencapsulation devices encapsulating the allogeneic cells, the trophoblasts, or the allogeneic cells and the trophoblasts. The allogeneic cells can be therapeutic cells. In various aspects, the allogeneic cells are islets or islet cells.

In various aspects of the placenta-mimicking device, the trophoblasts are primary cells isolated from placenta. Trophoblasts can be cytotrophoblasts (CT), syncytiotrophoblasts (ST), extravillous trophoblasts (EVT), or any combination thereof. In further aspects, the trophoblasts are human trophoblast-like cell lines selected from JEG3, JAR, and combinations thereof.

In various aspects, the trophoblasts are organoids comprising trophoblasts. For example, in various aspects, the trophoblasts are organoids comprising trophoblasts derived from a trophoblast-like cell line. In various aspects, the trophoblasts are organoids comprising trophoblasts wherein the organoids are prepared using a method comprising: (a) providing or having providing trophoblasts; (b) embedding the trophoblasts in a degradable hydrogel matrix comprising a 3D matrix and one or more adhesive ligands; and (c) incubating the trophoblasts under conditions and for a time sufficient for the trophoblasts to generate the organoids.

In various aspects, the hydrogel matrix in the placenta-mimicking device comprises poly(ethylene glycol) (PEG), agarose, alginate, or any combination thereof. In various aspects, the hydrogel matrix comprises alginate. In various aspects, the hydrogel matrix comprises maleimide-functionalized, 4-arm PEG cross-linked using protease-degradable peptides. In various aspects, the hydrogel matrix comprises a PEG hydrogel crosslinked with degradable crosslinkers, nondegradable crosslinker, or any combination thereof. In various aspects the degradable crosslinkers can be protease-degradable peptides selected from VPM, GDQ, GDQ-W, and any combination thereof. In further aspects, the degradable crosslinker can be a VPM degradable peptide.

In various aspects, the microencapsulation device in the placenta-mimicking device is nondegradable. In various aspects, the microencapsulation device is retrievable. In further aspects, the microencapsulation device encapsulates the trophoblasts and the allogeneic cells.

In various aspects, the placenta-mimicking device comprises a first macroencapsulation device encapsulating the trophoblasts and a second macroencapsulation device encapsulating the allogeneic cells, wherein a combination of the first macroencapsulation device and the second macroencapsulation device is operable to generate a vaccine when implanted into a subject in proximity to each other.

In various aspects, the macroencapsulation device comprises vascular endothelial growth factor (VEGF). In various aspects, the macroencapsulation device comprises RGD adhesive peptide. In various aspects, the macroencapsulation device comprises a nondegradable biocompatible hydrogel matrix. The hydrogel matrix can comprise poly(ethylene glycol) (PEG), agarose, alginate, or any combination thereof. For example, in various aspects, the hydrogel matrix comprises alginate. In further aspects, the hydrogel matrix comprises multi-arm PEG functionalized with bioorthogonal reactive groups.

In various aspects, the placenta-mimicking device comprises a macroencapsulation device comprising a PEG hydrogel matrix crosslinked using a nondegradable linker. In various aspects, the hydrogel matrix is operable to be crosslinked within an injection mold, and wherein the hydrogel macroencapsulation device is formed from the injection mold. In various aspects, the hydrogel matrix comprises maleimide-functionalized, 4-arm PEG crosslinked using a nondegradable linker. In various aspects, the macroencapsulation device comprises a 20 kDa 4-arm PEG hydrogel matrix crosslinked using a nondegradable linker. In various aspects, the nondegradable crosslinkers are protease-degradable peptides selected from DTT, PEG-DT, and any combination thereof.

In various aspects, the placenta-mimicking device comprises a hydrogel matrix functionalized with one or more adhesive peptides. In various aspects, the one or more adhesive peptide is collagen I, collagen IV, laminin, fibronectin, fibrinogen, osteopontin, plasminogen, vitronectin, arginylglycylaspartic acid (RGD), or any combination thereof. In various aspects, the one or more adhesive peptide is collagen IV, laminin, arginylglycylaspartic acid (RGD), or any fraction thereof, or any combination thereof. In various aspects, the adhesive peptide is carginylglycylaspartic acid (RGD).

In various aspects, the one or more adhesive peptides are 3, 4, 5, 6, 7, 8, 9 or 10 amino acids in length. In various aspects, the one or more adhesive peptide comprises an amino acid sequence selected from RGD, RDG, GFOGER, YIGSR, IKVAV, REDV, DGEA, VGVAPG, GRGDS, LDV, RGDV, PDSGR, RYVVLPR, LGTIPG, LAG, RGDS, RGDF, HHLGGALQAGDV, VTCG, SDGD, GREDVY, GRGDY, GRGDSP, VAPG, GGGGRGDSP, GGGGRGDY, FTLCFD, or any combination thereof.

In various aspects, the microencapsulation device in the placenta mimicking device comprises a spiral geometry, a wrinkled sheet geometry, a planar sheet geometry, a branched geometry, or a vascular geometry.

Also provided is another placenta-mimicking device comprising: (a) cells capable of producing insulin; (b) organoids comprising trophoblasts; and (c) one or more hydrogel matrix encapsulating the cells capable of producing insulin, the organoids, or the cells capable of producing insulin and the organoids, the hydrogel matrix comprising a 3D matrix structure comprising: (i) a crosslinked poly(ethylene glycol) (PEG), a hydrogel comprising maleimide-functionalized, 20 kDa 4-arm PEG cross-linked using a nondegradable peptide selected from DTT, PEG-DT, and any combination thereof; (ii) an RGD adhesive peptide; and (iii) vascular endothelial growth factor (VEGF). In various aspects, the cells capable of producing insulin produce insulin in a glucose-dependent manner.

Also provided is another placenta-mimicking device comprising: (a) organoids comprising trophoblasts, wherein the organoids are prepared using a method comprising: (i) providing or having provided trophoblasts; (ii) embedding the trophoblasts in a degradable hydrogel matrix comprising a 3D matrix and one or more adhesive ligands; and (iii) incubating the cells under conditions and for a time sufficient for the trophoblasts to generate organoids comprising the trophoblasts; (b) allogeneic cells; and (c) one or more macroencapsulation devices encapsulating the allogeneic cells, the organoids, or the allogeneic cells and the organoids.

In various aspects, the microencapsulation device of the placenta-mimicking device comprises a nondegradable biocompatible hydrogel matrix. In various aspects, the hydrogel matrix comprises poly(ethylene glycol) (PEG), agarose, alginate, or any combination thereof. For example, in various aspects, the hydrogel matrix comprises alginate. In various aspects, the hydrogel matrix comprises multi-arm PEG functionalized with bioorthogonal reactive groups.

In various aspects, the macroencapsulation device comprises a poly(ethylene glycol) (PEG) hydrogel matrix crosslinked using a nondegradable linker. In various aspects, the hydrogel matrix comprises maleimide-functionalized, 4-arm PEG crosslinked using a nondegradable linker. In further aspects, the macroencapsulation device comprises a 20 kDa 4-arm PEG hydrogel matrix crosslinked using a nondegradable linker. In various aspects, the nondegradable crosslinkers are protease-degradable peptides selected from DTT, PEG-DT, and any combination thereof.

Also provided is a method of preparing organoids comprising trophoblasts, the method comprising: (a) providing or having provided trophoblasts; (b) embedding the trophoblasts in a degradable hydrogel matrix comprising a 3D matrix and one or more adhesive ligands; and (c) incubating the cells under conditions and for a time sufficient for the trophoblasts to generate organoids comprising the trophoblasts.

In various aspects, the hydrogel matrix comprises poly(ethylene glycol) (PEG), agarose, alginate, or any combination thereof. For example, in various aspects, the hydrogel matrix comprises alginate. In various aspects, the hydrogel matrix comprises maleimide-functionalized, 4-arm PEG cross-linked using protease-degradable peptides. In various aspects, the hydrogel matrix comprises a PEG hydrogel crosslinked with degradable crosslinkers, nondegradable crosslinker, or any combination thereof. In degradable crosslinkers can be protease-degradable peptides selected from VPM, GDQ, GDQ-W, or any combination thereof. In various aspects the degradable crosslinker is a VPM degradable peptide.

In various aspects, the organoids are derived from primary trophoblasts, stem cells, from trophoblast-like cell lines selected from JEG3, JAR, and combinations thereof. In various aspects, the organoids are derived from primary trophoblasts. In various aspects, the organoids are derived from trophoblast-like cell lines such as JEG3, JAR, and combinations thereof. In various aspects, the organoids are derived from stem cells.

Also provided is a method of preventing and/or treating an immunological response to transplanted allogeneic cells in a subject in need thereof, the method comprising implanting a placenta-mimicking device, the placenta-mimicking device comprising: (a) allogeneic cells; (b) trophoblasts; and (c) one or more microencapsulation devices encapsulating the allogeneic cells, the trophoblasts, or the allogeneic cells and the trophoblasts.

In various aspects, the macroencapsulation device encapsulates the trophoblasts and the allogeneic cells. In further aspects, the placenta-mimicking device comprises a first macroencapsulation device encapsulating the trophoblasts and a second macroencapsulation device encapsulating the allogeneic cells, and the method comprises implanting the first macroencapsulation device and the second macroencapsulation device.

In various aspects, the first encapsulation device is implanted between about 1-30 days prior to implanting the second encapsulation device. In various aspects, the first and the second encapsulation devices are implanted in proximity to each other. In various aspects, the device is implanted in the omentum or subcutaneously.

Also provided is a method of treating diabetes Type I in a subject in need thereof, the method comprising implanting a placenta-mimicking device, the placenta-mimicking device comprising: (a) cells capable of producing insulin; (b) trophoblasts; and (c) one or more macroencapsulation devices encapsulating the cells capable of producing insulin, the trophoblasts, or the cells capable of producing insulin and the trophoblasts. In various aspects, the cells capable of producing insulin produce insulin in a glucose-dependent manner. In various aspects, the cells capable of producing insulin are islets or islet cells.

Also provided is a kit comprising one or more placenta-mimicking devices described herein for use in any method described herein.

Also provided is a use of one or more placenta mimicking devices described herein for preventing and/or treating an immunological response to transplanted allogeneic cells in a subject in need thereof or for treating diabetes Type I in a subject in need thereof.

In another aspect of the instant disclosure, an artificial placenta vaccine device is provided, comprising (a) trophoblasts, (b) donor cells; and (c) one or more microencapsulation devices encapsulating the donor cells, the trophoblasts, or the donor cells and the trophoblasts.

In various aspects, the donor cells are from a tissue donor or are from the same source as the donor tissue.

In various aspects, the trophoblasts are primary cells isolated from placenta. In various aspects, the trophoblasts are cytotrophoblasts (CT), syncytiotrophoblasts (ST), extravillous trophoblasts (EVT), or any combination thereof. In various aspects, the trophoblasts are human trophoblast-like cell lines selected from JEG3, JAR, and combinations thereof.

In various aspects, the trophoblasts are organoids comprising trophoblasts. In various aspects, the trophoblasts are organoids comprising trophoblasts derived from cultured primary cells isolated from placenta. In various aspects, the trophoblasts are organoids comprising trophoblasts, wherein the organoids are prepared using a method comprising: (a) providing or having provided trophoblasts; (b) embedding the trophoblasts in a degradable hydrogel matrix comprising a 3D matrix and one or more adhesive ligands; and (c) incubating the trophoblasts under conditions and for a time sufficient for the trophoblasts to generate the organoids.

In various aspects, the hydrogel matrix comprises poly(ethylene glycol) (PEG), agarose, alginate, or any combination thereof. In various aspects, the hydrogel matrix comprises alginate. In various aspects, the hydrogel matrix comprises maleimide-functionalized, 4-arm PEG cross-linked using protease-degradable peptides. In further aspects, the hydrogel matrix comprises a PEG hydrogel crosslinked with degradable crosslinkers, nondegradable crosslinker, or any combination thereof. In various aspects, the degradable crosslinkers are protease-degradable peptides selected from VPM, GDQ, GDQ-W, and any combination thereof. In various aspects, the degradable crosslinker is a VPM degradable peptide.

In various aspects, the macroencapsulation device is nondegradable. In various aspects, the macroencapsulation device is retrievable. In various aspects, the macroencapsulation device encapsulates the trophoblasts and the donor cells.

In various aspects, the placenta vaccine device comprises a first macroencapsulation device encapsulating the trophoblasts and a second macroencapsulation device encapsulating the donor cells, wherein a combination of the first macroencapsulation device and the second macroencapsulation device is operable to generate a vaccine when implanted into a subject in proximity to each other.

In various aspects, the macroencapsulation device comprises vascular endothelial growth factor (VEGF). In various aspects, the macroencapsulation device comprises RGD adhesive peptide.

In further aspects, the macroencapsulation device comprises a nondegradable biocompatible hydrogel matrix. The hydrogel matrix can comprise poly(ethylene glycol) (PEG), agarose, alginate, or any combination thereof. For example, the hydrogel matrix can comprise alginate. In various aspects, the hydrogel matrix comprises multi-arm PEG functionalized with bioorthogonal reactive groups.

In various aspects, the macroencapsulation device comprises a poly(ethylene glycol) (PEG) hydrogel matrix crosslinked using a nondegradable linker.

In various aspects, the hydrogel matrix is operable to be crosslinked within an injection mold, and wherein the hydrogel macroencapsulation device is formed from the injection mold. In various aspects, the hydrogel matrix comprises maleimide-functionalized, 4-arm PEG crosslinked using a nondegradable linker. In various aspects, the macroencapsulation device comprises a 20 kDa 4-arm PEG hydrogel matrix crosslinked using a nondegradable linker. In various aspects, the nondegradable crosslinkers are protease-degradable peptides selected from DTT, PEG-DT, and any combination thereof.

In various aspects, the hydrogel matrix is functionalized with one or more adhesive peptides. In various aspects, the adhesive peptide is collagen I, collagen IV, laminin, fibronectin, fibrinogen, osteopontin, plasminogen, vitronectin, arginylglycylaspartic acid (RGD), or any combination thereof. For example, the adhesive peptide can be collagen IV, laminin, arginylglycylaspartic acid (RGD), or any fraction thereof, or any combination thereof. In various aspects, the adhesive peptide is carginylglycylaspartic acid (RGD).

In various aspects, the one or more adhesive peptides are 3, 4, 5, 6, 7, 8, 9 or 10 amino acids in length. In various aspects, the one or more adhesive peptide consist of or comprises an amino acid sequence selected from RGD (SEQ ID NO: 1), RDG (SEQ ID NO: 2), GFXGER, wherein X is hydroxyproline (SEQ ID NO: 3; also referred to elsewhere herein as GFOGER), YIGSR (SEQ ID NO: 4), IKVAV (SEQ ID NO: 5), REDV (SEQ ID NO: 6), DGEA (SEQ ID NO: 7), VGVAPG (SEQ ID NO: 8), GRGDS (SEQ ID NO: 9), LDV (SEQ ID NO: 10), RGDV (SEQ ID NO: 11), PDSGR (SEQ ID NO: 12), RYVVLPR (SEQ ID NO: 13), LGTIPG (SEQ ID NO: 14), LAG (SEQ ID NO: 15), RGDS (SEQ ID NO: 16), RGDF (SEQ ID NO: 17), HHLGGALQAGDV (SEQ ID NO: 18), VTCG (SEQ ID NO: 19), SDGD (SEQ ID NO: 20), GREDVY (SEQ ID NO: 21), GRGDY (SEQ ID NO: 22), GRGDSP (SEQ ID NO: 23), VAPG (SEQ ID NO: 24), GGGGRGDSP (SEQ ID NO: 25), GGGGRGDY (SEQ ID NO: 26), FTLCFD (SEQ ID NO: 27), or any combination thereof.

In various aspects, the microencapsulation device comprises a spiral geometry, a wrinkled sheet geometry, a planar sheet geometry, a branched geometry, or a vascular geometry.

Also provided herein is another artificial placenta vaccine device comprising: (a) donor cells; (b) organoids comprising trophoblasts; and (c) one or more hydrogel matrix encapsulating the donor cells, the organoids, or the donor cells and the organoids, the hydrogel matrix comprising a 3D matrix structure comprising: (i) a crosslinked poly(ethylene glycol) (PEG) hydrogel comprising maleimide-functionalized, 20 kDa 4-arm PEG cross-linked using a combination of protease-degradable peptides and nondegradable crosslinkers; (ii) RGD adhesive peptide; and (iii) vascular endothelial growth factor (VEGF).

In another aspect, another artificial placenta vaccine device is provided, comprising: (a) organoids comprising trophoblasts wherein the organoids are prepared using a method comprising: (i) providing or having provided trophoblasts; (ii) embedding the trophoblasts in a degradable hydrogel matrix comprising a 3D matrix and one or more adhesive ligands; and (iii) incubating the cells under conditions and for a time sufficient for the trophoblasts to generate organoids comprising the trophoblasts; (b) donor cells; and (c) one or more macroencapsulation devices encapsulating the donor cells, the organoids, or the donor cells and the organoids.

In various aspects, the macroencapsulation device comprises a nondegradable biocompatible hydrogel matrix. In various aspects, the hydrogel matrix comprises poly(ethylene glycol) (PEG), agarose, alginate, or any combination thereof. In various aspects, the hydrogel matrix comprises multi-arm PEG functionalized with bioorthogonal reactive groups.

In various aspects, the macroencapsulation device comprises a poly(ethylene glycol) (PEG) hydrogel matrix crosslinked using a nondegradable linker. In various aspects, the hydrogel matrix comprises maleimide-functionalized, 4-arm PEG crosslinked using a nondegradable linker. In further aspects, the macroencapsulation device comprises a 20 kDa 4-arm PEG hydrogel matrix crosslinked using a nondegradable linker. In various aspects, the nondegradable crosslinkers are protease-degradable peptides selected from DTT, PEG-DT, and any combination thereof.

In various aspects, the degradable hydrogel matrix comprises poly(ethylene glycol) (PEG), agarose, alginate, or any combination thereof. In various aspects, the degradable hydrogel matrix comprises alginate. In various aspects, the degradable hydrogel matrix comprises maleimide-functionalized, 4-arm PEG cross-linked using protease-degradable peptides. In further aspects, the degradable hydrogel matrix comprises a PEG hydrogel crosslinked with degradable crosslinkers, nondegradable crosslinker, or any combination thereof. In various aspects, the degradable crosslinkers are protease-degradable peptides selected from VPM, GDQ, GDQ-W, and any combination thereof. In various aspects, the degradable crosslinker is a VPM degradable peptide.

In various aspects, a method of preparing organoids comprising trophoblasts is provided, the method comprising: (a) providing or having provided trophoblasts; (b) embedding the trophoblasts in a degradable hydrogel matrix comprising a 3D matrix and one or more adhesive ligands; and (c) incubating the cells under conditions and for a time sufficient for the trophoblasts to generate organoids comprising the trophoblasts.

In various aspects, the degradable hydrogel matrix comprises poly(ethylene glycol) (PEG), agarose, alginate, or any combination thereof. In various aspects, the degradable hydrogel matrix comprises alginate. In various aspects, the degradable hydrogel matrix comprises maleimide-functionalized, 4-arm PEG cross-linked using protease-degradable peptides. In various aspects, the degradable hydrogel matrix comprises a PEG hydrogel crosslinked with degradable crosslinkers, nondegradable crosslinker, or any combination thereof. In various aspects, the degradable crosslinkers are protease-degradable peptides selected from VPM, GDQ, GDQ-W, and any combination thereof. In various aspects, the degradable crosslinker is a VPM degradable peptide.

In various aspects, the organoids are derived from primary trophoblasts, stem cells, from trophoblast-like cell lines selected from JEG3, JAR, and combinations thereof. In various aspects, the organoids are derived from primary trophoblasts. In various aspects, the organoids are derived from trophoblast-like cell lines such as JEG3, JAR, and combinations thereof. In various aspects, the organoids are derived from stem cells.

A method of preventing and/or treating an immunological response to a transplanted tissue in a subject in need thereof is also provided, the method comprising: (a) implanting any artificial placenta vaccine device described herein and (b) transplanting donor tissue into the subject concurrently with or prior to implanting the artificial placenta vaccine device.

In various aspects, the microencapsulation device encapsulates the trophoblasts and the donor cells. In various aspects, the artificial placenta vaccine device used in the method provided herein, comprises a first macroencapsulation device encapsulating the trophoblasts and a second macroencapsulation device encapsulating the donor cells, and the method comprises implanting the first macroencapsulation device and the second macroencapsulation device.

In various aspects the first encapsulation device is implanted between about 1-30 days prior to implanting the second encapsulation device. In further aspects, the first and the second encapsulation devices are implanted in proximity to each other.

In various aspects, the artificial placenta vaccine device is implanted in the omentum or subcutaneously.

In another aspect, an immunosuppression-free tissue transplantation method is provided, the method comprising: (a) implanting an artificial placenta vaccine device as provided herein into the subject; and (b) transplanting donor tissue into the subject concurrently with or prior to implanting the artificial placenta vaccine device.

Also provided is a kit comprising one or more artificial placenta vaccine devices described herein for use in any method described herein.

Also provided is a use for one or more artificial placenta vaccine devices described herein for transplanting donor tissue into a subject in need thereof in the absence of chronic systemic immunosuppression.

### BRIEF DESCRIPTION OF THE FIGURES

The patent or application file contains at least one drawing executed in color. Copies of this patent or patent application publication with color drawing(s) will be provided by the Office upon request and payment of the necessary fee. At least FIGS. 6B-6D cannot be reproduced in black and white because the color is used to depict multiple color gradients that cannot be reproduced in black and white

FIG. 1 is a visual summary of utilizing trophoblasts and synthetic hydrogel matrices to investigate trophoblast immunology and induce tolerance toward allogeneic grafts.

FIG. 2A illustrates synthetic hydrogel matrices for cell encapsulation. The figure shows that poly(ethylene glycol) (PEG)-based hydrogels leveraging bioorthogonal chemistry can be laden with bioactive signals (e.g. RGD, VEGF), and crosslinked with nondegradable linker.

FIG. 2B illustrates synthetic hydrogel matrices for cell encapsulation. The figure shows a PEG hydrogels for islet microencapsulation and

FIG. 2C illustrates synthetic hydrogel matrices for cell encapsulation. The figure shows a PEG hydrogel for islet macroencapsulation. Scale bar=200 μm. Gel, diameter=10 mm

FIG. 3A shows Architecture of placental villi, demonstrating the typical location of cytotrophoblasts (CT), synctiotrophoblasts (ST), and extravillous trophoblasts (EVT).

FIG. 3B shows microvilli geometry of a placenta-on-a-chip model that could help interrogate the role of individual trophoblast cell types on localized induction of tolerance toward fetal allografts (extravillous trophoblast (EVT), cytotrophoblast (CT), and syncitotrophoblast (ST)).

FIG. 3C show a simplified geometry of a placenta-on-a-chip model that could help interrogate the role of individual trophoblast cell types on localized induction of tolerance toward fetal allografts (extravillous trophoblast (EVT), cytotrophoblast (CT), and syncitotrophoblast (ST)).

FIG. 4A illustrates probing the immunomodulation of trophoblasts in engineered synthetic hydrogel matrices. The figure illustrates the influence of cell subtype and placenta architecture on trophoblast tolerogenic capacity via synthetic hydrogel constructs

FIG. 4B illustrates probing the immunomodulation of trophoblasts in engineered synthetic hydrogel matrices. The figure illustrates investigation of the soluble and matrix-bound cues for their impact on CT differentiation and long-term behavior in 3D synthetic hydrogel matrices.

FIG. 5A shows the impact of encapsulation device geometry on islet oxygenation. Vascularized micro- or macroencapsulated islets in the EFP or omentum, respectively.

FIG. 5B shows the impact of encapsulation device geometry on islet oxygenation. Finite element modeling of macroencapsulation device oxygen gradients in vivo illustrated suboptimal oxygen levels, with or without VEGF delivery. Scale bar 200 μm.

FIG. 6A illustrate hydrogel macroencapsulation device design and fabrication. An injection molding device to fabricate complex 3D hydrogel macroencapsulation devices designed for optimal cell oxygenation at high cell densities.

FIG. 6B shows oxygen consumption rates in traditional capsule encapsulation.

FIG. 6C shows oxygen consumption rates in a “spiral” macroencapsulation device.

FIG. 6D shows oxygen consumption rates in a “sheet” macroencapsulation.

FIG. 6E show that due to rapid prototyping via 3D printing of the injection molds, macroencapsulation devices can be augmented to meet design needs.

FIG. 6F shows example schemes to generate hydrogels in an injection molding strategy.

FIG. 7 illustrates methods of localizing the presentation of immunomodulatory factors.

FIG. 8 illustrates the viability of JEG-3 extravillous trophoblast-like cell line in PEG hydrogels with varying crosslinkers and RGD adhesion ligand through confocal imaging on days 1, 3 and 7. Cells were stained with Calcein AM (green—live cells) and ethidium homodimer (magenta—dead cells). The cells create cluster structures over time, more so in hydrogels with degradable crosslinkers (VPM, GDQ, and GDQ-W) versus the nondegradable hydrogels (DTT, PEG-DT), where more cell death is also seen. A half-half mix of degradable crosslinkers was also tested that creates bigger structures than fully nondegradable hydrogels. Scale bar=200 um.

FIG. 9 illustrates the viability of JAR villous trophoblast-like cell line in PEG hydrogels with varying crosslinkers and RGD adhesion ligand through confocal imaging on days 1, 3 and 7. Cells were stained with Calcein AM (green—live cells) and ethidium homodimer (magenta—dead cells). The cells create cluster structures over time, more so in hydrogels with degradable crosslinkers (VPM, GDQ, and GDQ-W) versus the nondegradable hydrogels (DTT, PEG-DT), where more cell death is also seen. A half-half mix of degradable crosslinkers was also tested that creates bigger structures than fully nondegradable hydrogels. Scale bar=200 um

FIG. 10 illustrates the viability of JEG-3 extravillous trophoblast-like cell line in PEG hydrogels with varying adhesion ligands and VPM crosslinker, as well as these cells in a Matrigel control, through confocal imaging on days 1, 3 and 7. Cells were stained with Calcein AM (green—live cells) and ethidium homodimer (magenta—dead cells). The cells create cluster structures over time in all PEG hydrogels using fibronectin (RGD, scrambled—RDG), collagen (GFOGER), and laminin (YIGSR, IKVAV) motifs. Cells did not create structures in the Matrigel control. Scale bar=200 um

FIG. 11 illustrates the viability of JAR villous trophoblast-like cell line in PEG hydrogels with varying adhesion ligands and VPM crosslinker, as well as these cells in a Matrigel control, through confocal imaging on days 1, 3 and 7. Cells were stained with Calcein AM (green—live cells) and ethidium homodimer (magenta—dead cells). The cells create cluster structures over time in all PEG hydrogels using fibronectin (RGD, scrambled—RDG), collagen (GFOGER), and laminin (YIGSR, IKVAV) motifs. Cells did not create structures in the Matrigel control. Scale bar=200 um

FIG. 12 illustrates the metabolism functionality of JEG-3 extravillous and JAR villous trophoblast-like cell lines in PEG hydrogels with varying crosslinkers and RGD adhesion ligand, as well as a Matrigel & 2D culture controls, through the alamarBlue assay on day 7. JEG-3 and JAR cells cultured in PEG hydrogels with degradable crosslinkers (VPM, GDQ, GDQ-W) and in 2D show a significant increase in metabolic activity over Matrigel controls. JAR cells cultured in nondegradable (DTT, PEG-DT) hydrogels also show a significant increase in metabolic activity over Matrigel; however, JEG-3 cells do not. Data shown is mean±SEM. n=10 gels from 1-3 independent experiments. ****=p<0.0001, **=p<0.01, ns=not significant

FIG. 13 illustrates the metabolism functionality of JEG-3 extravillous and JAR villous trophoblast-like cell lines in PEG hydrogels with varying adhesion ligands and VPM crosslinker, as well as a Matrigel & 2D culture controls, through the alamarBlue assay on day 7. JEG-3 and JAR cells cultured in PEG hydrogels and in 2D show a significant increase in metabolic activity over Matrigel controls. Data shown is mean±SEM. n=10-12 gels from 1-3 independent experiments. ****=p<0.0001, ***=p<0.001, **=p<0.01

FIG. 14 illustrates the secretion of beta human chorionic gonadotropin (hCGβ) by JEG-3 extravillous and JAR villous trophoblast-like cell lines in PEG hydrogels with varying adhesion ligands and VPM crosslinker, as well as a Matrigel & 2D culture controls, through enzyme-linked immunosorbent assays (ELISA) on day 7. JEG-3 cells cultured in PEG hydrogels with RDG and JAR cells cultured in PEG hydrogels with all the tested adhesion ligands and in 2D show a significant increase in hCGβ secretion over Matrigel controls. Data shown is mean±SEM. n=4-9 gels from 1-4 independent experiments. ****=p<0.0001, ***=p<0.001, ns=not significant

FIG. 15 illustrates the secretion of matrix metalloproteinase-2 (MMP2) by JAR villous trophoblast-like cell lines in PEG hydrogels with varying crosslinkers and RGD adhesion ligand, as well as a Matrigel & 2D culture controls, through enzyme-linked immunosorbent assays (ELISA) on day 7. JAR cells cultured in PEG hydrogels with degradable crosslinkers (VPM, half DTT-half VPM, GDQ, and GPQ-W) and in 2D show a significant increase in MMP2 secretion over Matrigel controls. JAR cells cultured in nondegradable crosslinkers (DTT, PEG-DT) did not show a significant increase in MMP2 secretion. Data shown is mean±SEM. n=8 gels from 2 independent experiments. *****=p<0.0001, ***=p<0.001, ns=not significant.

FIG. 16 illustrates the secretion of matrix metalloproteinase-2 (MMP2) by JAR villous trophoblast-like cell lines in PEG hydrogels with varying adhesion ligands and VPM crosslinker, as well as a Matrigel & 2D culture controls, through enzyme-linked immunosorbent assays (ELISA) on day 7. JAR cells cultured in PEG hydrogels with RGD, RDG, GFOGER, and YIGSR and in 2D show a significant increase in MMP2 secretion over Matrigel controls. Data shown is mean±SEM. n=8 gels from 2 independent experiments. ****=p<0.0001, ***=p<0.001, ns=not significant

### DETAILED DESCRIPTION

The present disclosure relates to transplantation devices, and methods of using the devices for facilitating allogeneic transplantation of tissue or cells to a recipient in the absence of immunosuppression. The devices and methods of the present disclosure can eliminate the need for chronic systemic immunosuppression of the natural immune response in the recipient, thereby avoiding transplant rejection and dramatically reducing the long-term risks associated chronic systemic immunosuppressive drugs.

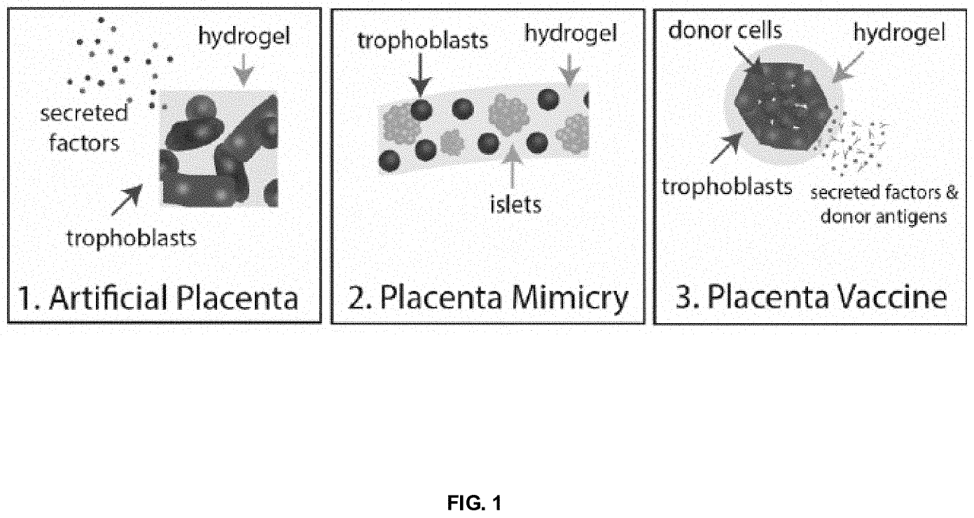

The current disclosure is based in part on the discovery that elements mediating the condition of placental pregnancy, which is the only known physiological scenario of allogeneic tissue tolerance, can be successfully leveraged to create an artificial construct useful for inducing immune tolerance in transplantation. This disclosure harnesses the tolerogenic immunomodulation mechanisms of trophoblasts and their capacity to induce tolerance in allogeneic graft transplantation to create the following (FIG. 1): (1) a synthetic hydrogel-based artificial placenta (the transplantation device), (2) methods of inducing placental mimicry for immunosuppression-free cell transplantation using the transplantation device, and (3) a placenta “vaccine” for immunosuppression-free organ transplantation using the transplantation device.

A transplantation device as described herein comprises allogeneic cells and immunomodulatory trophoblasts encapsulated in a macroencapsulation device. The transplantation device can instruct immune tolerance toward allogeneic grafts such as transplanted cells or tissue in a recipient. The disclosed transplantation devices have the advantage that they can be safely delivered and removed, if necessary, from the patient. By encapsulating trophoblasts within a fully retrievable, nondegradable hydrogel device, the risks associated with transplantation of an unpredictable and potentially invasive cell type are reduced.

### I. Device

Thus, one aspect of the present disclosure encompasses a transplantation device comprising one or more hydrogel macroencapsulation devices encapsulating allogeneic cells, trophoblasts, or allogeneic cells and trophoblasts. The macroencapsulation device is described in detail in Section I(a), the trophoblasts are described in Section I(b), and the allogeneic cells are described in Section I(c).

### (a) Hydrogel Macroencapsulation Device

A transplantation device of the instant disclosure comprises a hydrogel macroencapsulation device encapsulating trophoblasts, allogeneic (donor) cells, or any combination of trophoblasts and allogeneic cells. For example, a single macroencapsulation device can encapsulate the trophoblasts and the allogenic cells. Alternatively, a first macroencapsulation device may encapsulate trophoblasts, and a second macroencapsulation device may encapsulate allogenic cells, wherein the first macroencapsulation device and the second macroencapsulation device are administered in combination to achieve immunosuppression as described herein.

The hydrogel macroencapsulation device houses and maximizes cell viability of transplanted allogeneic cells in a recipient, shields the cells from direct antigen recognition by the recipient immune system, and provides for localized delivery or presentation of tolerogenic factors produced by the tolerogenic trophoblasts. The disclosed hydrogel macroencapsulation devices are also nondegradable, thereby providing the advantage that they can be safely delivered to the patient and removed from the patient, if necessary. In some aspects, hydrogels and a macroencapsulation device of the instant disclosure are as described in U.S. patent application Ser. No. 16/951,452, the disclosure of which is herein incorporated by reference in its entirety.

As some allogeneic cells such as islets, exhibit oxygen consumption rates up to 1000-fold higher than other cell types, the primary limitation of macroencapsulation devices is adequate oxygenation of encapsulated islets due to isolation from high oxygen vascular tissue in order to prevent immune recognition. As such, the macroencapsulation devices are designed for optimal geometry with respect to oxygen distribution, prioritizing device designs which minimize distance between islet and vascular tissue, thereby maximizing device oxygenation. The macroencapsulation devices of the instant disclosure comprise a geometry for maximal oxygenation of encapsulated cells and for free diffusion of trophoblast-secreted tolerogenic particles as large as exosomes. Further, the hydrogel macroencapsulation devices are nondegradable, and are designed for delivery to defined transplant sites and for retrievability to maximize device safety.

The hydrogel macroencapsulation device maximizes cell viability and function through optimization of geometry and encapsulating material and prioritizes facile device implementation in the clinic and automation and scale-up by fabrication via injection molding. The hydrogel macroencapsulation device can have a complex geometry, created by using an injection mold device or other methods of manufacture. The hydrogel macroencapsulation devices readily accommodate either homogeneous co-encapsulation of cells, or compartmentalized encapsulation depending on the optimal conformation of the device.

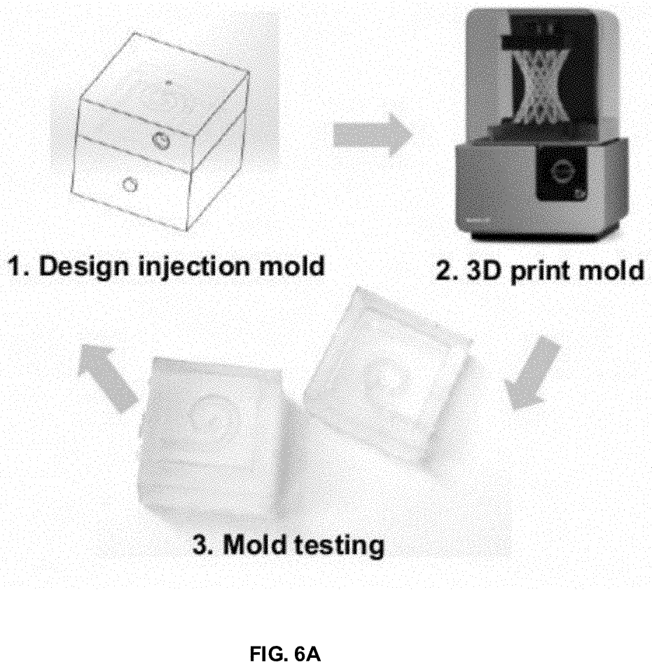

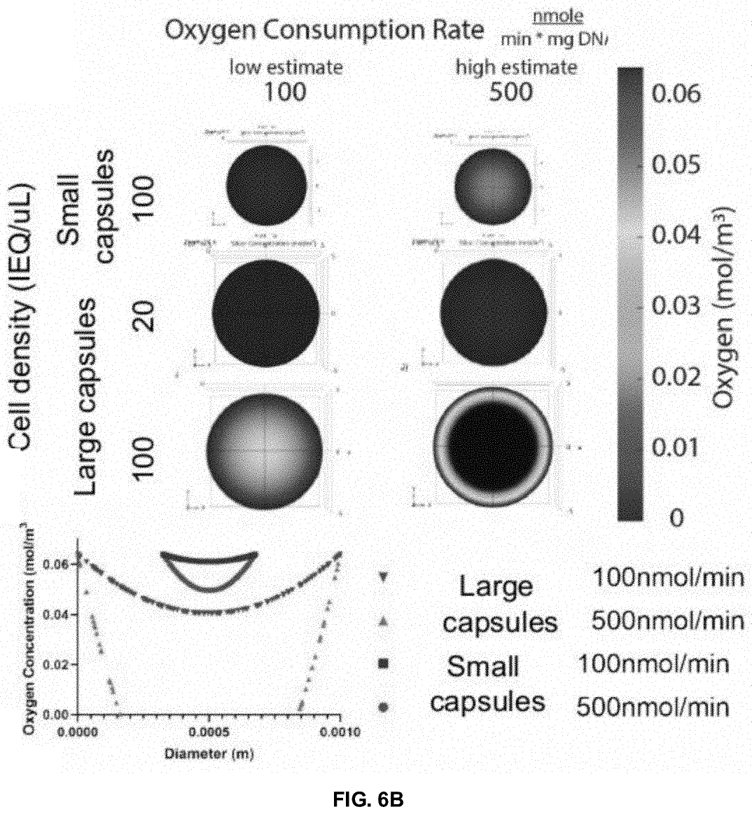

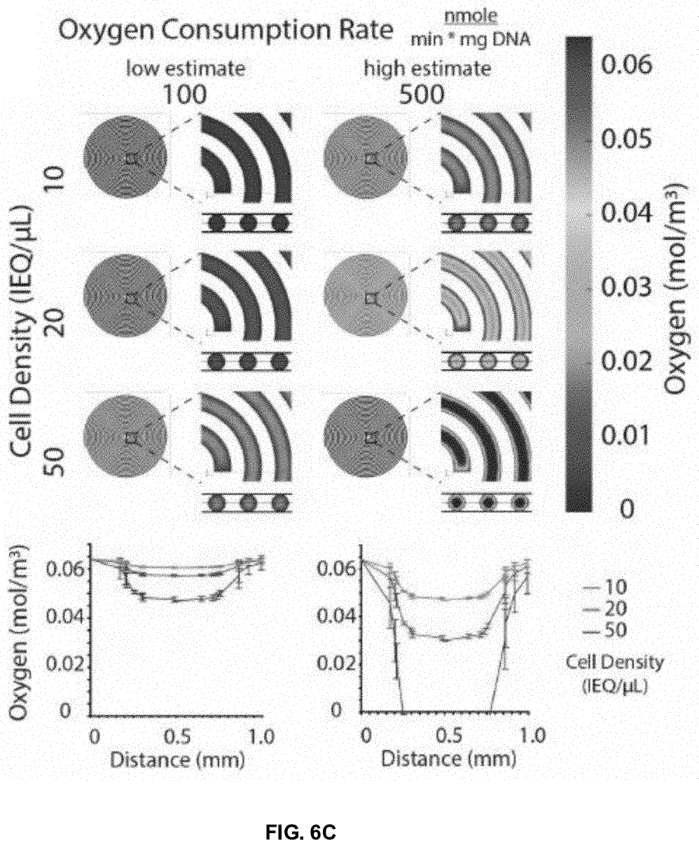

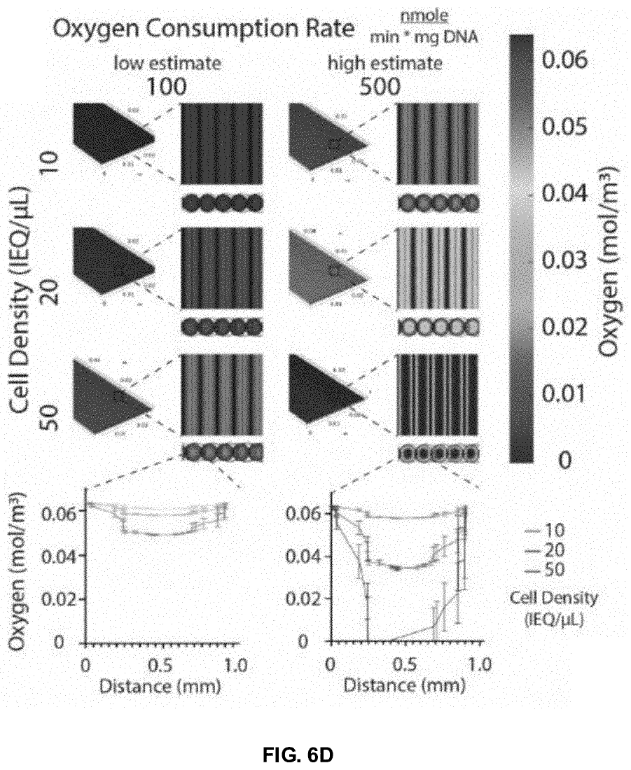

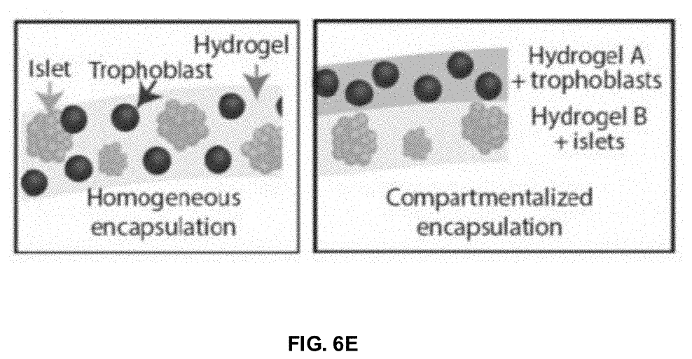

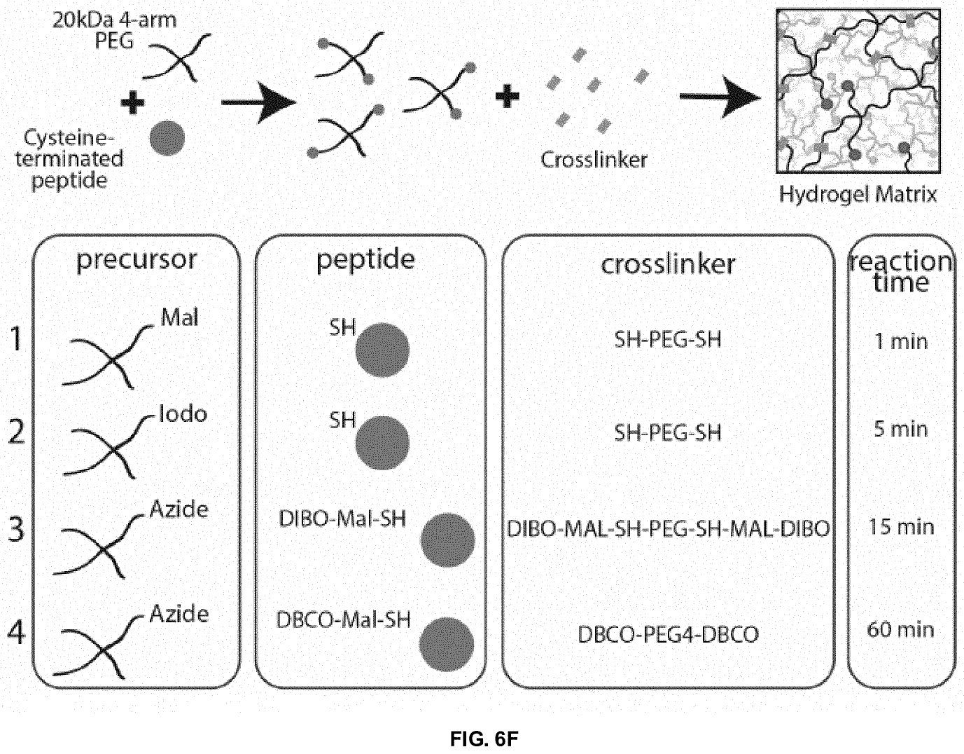

A number of different device geometries are contemplated by the present disclosure. For instance, the transplantation device can be a traditional capsule encapsulation device, a 3D printed device, a sheet macroencapsulation device, or any combination thereof. The hydrogel macroencapsulation device can have any geometry that reduces oxygen diffusion distances within the device sufficiently to preserve and maintain cell viability and function. For example, geometric device designs can be selected based on performance in finite element modeling of device oxygen profile. The hydrogel macroencapsulation device can have a 3-dimensional (3D) structure comprising channels. As seen in FIG. 6A, the geometry of the hydrogel macroencapsulation device can be formed by a channel in an injection mold device shaped into the geometry such that the diffusion distance through the hydrogel is minimized.

The hydrogel encapsulation device can be a hydrogel matrix. Biocompatible synthetic or natural hydrogels can be used to form the hydrogel macroencapsulation device. Non-limiting examples of hydrogels suitable for forming the macroencapsulation device include hydrogels prepared using hydrophilic biocompatible polymers such as poly(ethylene glycol) (PEG), polyacrylic acid, povidone, cellulose, agarose, gelatin, elastin, elastin-like polypeptides (ELP), collagen (any type of collagen or a mixture thereof), hyaluronic acid (HA), tropoelastin, fibronectin, laminin, chitosan, poly(ethylene sebacate) (PGS), poly(lactic acid) (PLA), vitronectin, poly-1-lysine, fibrin glue, gels made by decellularization of engineered and/or natural tissues, proteoglycans, alginate, polyglycolic acid (PGA), poly-caprolactone (PCL), polyvinyl alcohol (PVA), methyl methacrylate, poly(methyl methacrylate) (PMMA), poly(2-hydroxyethyl methacrylate) (PolyHEMA), poly(glycerol sebacate), polyurethanes, poly(isopropylacrylamide), poly(N-isopropylacrylamide), or any combination thereof. In some aspects, hydrogels for forming the macroencapsulation device include poly(ethylene glycol) (PEG), agarose, alginate, or any combination thereof. In one aspect, alginate is used to form the encapsulation device. In at least one aspect, polyethylene glycols (PEGs) are used due to their tenability and reproducibility in manufacturing and scaling. Typical synthetic hydrogel matrices are designed for in situ use, necessitating fast-polymerizing polymers. However, hydrogels with slower polymerization rates can be beneficial in an injection molding scheme, where sufficient time is required to mix components and inject into the mold. Advantages over faster-crosslinking hydrogels include greater uniformity and homogeneity in final hydrogel product crosslinking, expected to result in greater consistency of performance in vivo. In some examples, the macroencapsulation device can utilize a PEG bioorthogonal reaction scheme, which has high biocompatibility and crosslinks under conditions such that it can be used with the injection mold.

Table 1 provides a library of compatible hydrogel reaction schemes that can be deployed within the injection mold. In some aspects, hydrogel components can include (1) multi-arm PEG macromer, in the range of 10-50 kDa, functionalized with bioorthogonal reactive groups (Table 1), (2) bioactive molecules, peptides and proteins to support cell function and viability, including any bioactive factors that can be bound to the matrix, and (3) crosslinker(s) with reactive groups corresponding to the bioorthogonal reacted groups listed in Table 1, with a nondegradable spacer (PEG 0.5-10 kDa).

In some aspects, the hydrogel matrix is a 3D hydrogel matrix. In an aspect, the hydrogel matrix can be a poly(ethylene glycol) based hydrogel (PEG-based hydrogel). The hydrogel matrix can be crosslinked. In some aspects, the hydrogel matrix can be crosslinked with a nondegradable linker. Non-limiting examples of nondegradable crosslinkers are selected from DTT, PEG-DT, and any combination thereof.

TABLE 1

| Reactive group | | Abbreviation |
|---|---|---|
| | Bioorthogonal reactive group | |
| tetrazine | norbonene | NB |
| | (E)-cyclooct-4-enol | TCO |
| azide | dibenzocyclooctyne | DBCO |
| | azidodibenzocyclooctyne | ADIBO |
| | dibenzoazacyclooctyne | DIBAC |
| | difluorocyclooctyne 2 | DIFO2 |
| | difluorocyclooctyne 3 | DIFO3 |
| | bicyclononyne | BCN |
| thiol | maleimide | MAL |
| | iodoacetamide | IODO |
| | Polymer backbone | |
| | 4-arm poly(ethylene glycol), 10-80 kDa | |
| | Alginate | |
| | Agarose | |

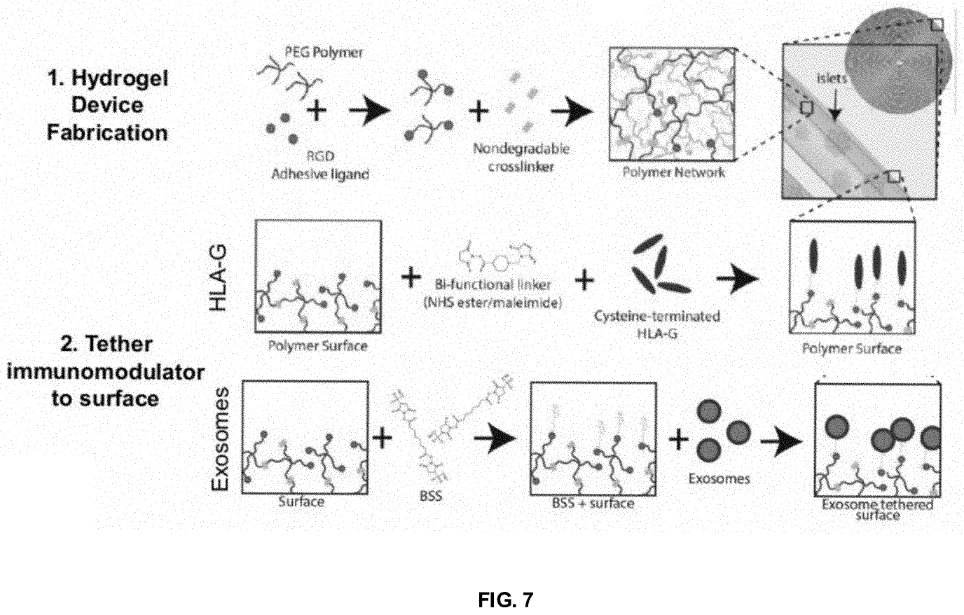

In some aspects, the hydrogel matrix can comprise one or more peptides and/or bioactive signals. an aspect, one or more of the peptides and/or bioactive signals can be banded to one or more components of the hydrogel matrix. In an aspect, one or more of the peptides and/or bioactive signals can be irreversibly bonded to one or more components of the hydrogel matrix. In an aspect, one or more of the peptides and/or bioactive signals can be reversibly bonded to one or more components of the hydrogel matrix. In some aspects, one or more of the peptides and/or bioactive signals can be soluble in the hydrogel matrix. The one or more of the peptides and/or bioactive signals can be added to the hydrogel matrix before crosslinking or after crosslinking. In some aspects, the bioactive signal can be localized in and/or on the encapsulation device. For example, FIG. 7 shows several methods of localizing bioactive signals, etc. utilizing trophoblast exosomes and/or HLA-G. By way of non-limiting example, the one or more peptides can be an adhesive peptide, such as but not limited to collagen I, collagen IV, laminin, fibronectin, fibrinogen, osteopontin, plasminogen, vitronectin, arginylglycylaspartic acid (RGD), or any combination thereof. In some aspects, an adhesive peptide can consist of or comprise any of the following amino acid sequences: RGD, RDG, GFOGER, YIGSR IKVAV, REDV, DGEA, VGVAPG, GRGDS, LDV, RGDV, PDSGR, RYVVLPR, LGTIPG, LAG, RGDS, RGDF, HHLGGALQAGDV, VTCG, SDGD, GREDVY, GRGDY, GRGDSP, VAPG, GGGGRGDSP, GGGGRGDY, or FTLCFD. An adhesive peptide can be 3, 4, 5, 6, 7, 8, 9 or 10 amino acids in length.

By way of non-limiting example, the one or more bioactive signals, can include one or more of: growth factors, angiogenic agents, hormones, or any combination thereof. Suitable growth factors can include, but are not limited to, epithelial growth factor (EGF). FIG. 6F provides an aspect of a crosslinking reaction and reagents that can be used to generate the hydrogels. acidic or basic fibroblast growth factor (FGF), vascular endothelial growth factor (VEGF), hepatocyte growth factor (HGF), heparin binding growth factor (HGBF), insulin-like growth factors (IGFs), transforming growth factor (TGF), nerve growth factor (NGF), muscle morphogenic factor, placental growth factor (PIGF) and platelet derived growth factor (PDGF). Suitable hormones can include, but are not limited to, Gonadotropin-releasing hormone (GnRH), human chorionic gonadotropin (hCG), insulin, estradiol, progesterone, follicle-stimulating hormone (FSH), and luteinizing hormone (LH). In some aspects, the angiogenic agent is vascular endothelial growth factor (VEGF).

In some aspects, the one or more bioactive signal is capable of influencing trophoblast differentiation. For instance, bioactive signals can be used in methods of preparing trophoblast organoids as described in Section IV. Bioactive signals capable of influencing trophoblast differentiation include but are not limited to the following: transcription factors, hormones, growth factors, cytokines, microRNAs (miRNA), or any combination thereof. In some aspects, a transcription factor can be one or more hypoxia-inducible factors (HIFs). In other aspects, a hormone can be gonadotropin-releasing hormone (GnRH), hCG, insulin, progesterone, estrogen, or a combination thereof. In some other aspects, a growth factor can be vascular endothelial growth factor (VEGF), transforming growth factor (TGF)-β, fibroblast growth factor (FGF), hepatocyte growth factor (HGF), epidermal growth factor (EGF), heparin-binding EGF (HB-EGF), insulin-like growth factors (IGFs), or a combination thereof. In some aspects, a cytokine can be macrophage-colony stimulating factor (M-CSF), leukemia inhibitory factor (LIF), an interleukin (IL), tumor necrosis factor (TNF), a chemokine (C-X-C motif) ligand, or a combination thereof. In some aspects, a miRNA can be miRNA-155, miR-141, miR-378a-5p, miR-21, miR-34a, miR-29b, miR-210, miR-195, miR-376c, miR-378-5p, miR-20a, miR-20b, miR-675, miR-148a, miR-152, miR-518c, or a combination thereof. In some aspects, the one or more bioactive signals can be one or more immunomodulatory signals. In some aspects, the one or more immunomodulatory signals are synergistic.

In some aspects, the hydrogel of the macroencapsulation device can include a vasculogenic hydrogel to encourage vascularization at the macroencapsulation device surface. Maximal oxygenation within the hydrogel macroencapsulation device is dependent upon the oxygen levels of tissue at the surface of the device. The higher the density of oxygen-rich vascular networks at the surface of the device, the higher the oxygenation within the device. Therefore, the configuration of this invention can include a coating of a vasculogenic degradable hydrogel at the device surface upon implantation.

Methods of fabricating a hydrogel macroencapsulation device of the instant disclosure can and will vary depending on the conformation of the device as well as the donor cells, the trophoblasts, and the intended use of the device among other factors Non-limiting examples of fabrication methods suitable for manufacturing a device include 3D printing, injection molding, or a combination thereof.

In some aspects, the hydrogel macroencapsulation device is fabricated using an injection molding strategy. In some aspects, the hydrogel macroencapsulation device is fabricated via 3D printing. To generate hydrogels in an injection molding strategy, synthetic hydrogel crosslinking reaction with kinetics that allow sufficient time to inject components into the mold prior to gelation is required.

### (b) Immunomodulatory Trophoblasts

Strategies disclosed herein utilize trophoblasts which are tolerogenic cells, the primary physiological role of which is the sustained and persistent maintenance of tolerance toward allogeneic tissue. In placental pregnancy, semi-allogeneic and fully allogeneic conceptus are protected from immune response by the placenta, an organ consisting of various types of trophoblasts, and which physically isolates the fetus from the mother. While the trophoblasts are derived from the fetus, they lack the antigens to stimulate immune response via direct antigen recognition, forming an immunologically inert barrier to immune cells. Additionally, fetal-derived shed antigens avoid maternal immune activation via the indirect antigen recognition pathway due to trophoblast-secreted tolerogenic factors. This diverse array of secreted factors induces ubiquitous, paternal antigen-specific maternal immune cell tolerance and anergy. Last, evidence of long-term chimerism between mother and fetus, wherein fetal cells persist in the mother for years post parturition, as well as documented immune hyporesponsiveness and tolerance toward paternal antigens in multiparous females, point to their potential use as a cell-based tolerogenic therapy.

As use herein, the term “tolerogenic” when used to describe a cell refers to the immunosuppressive properties of the cell obtained by priming the immune system into tolerogenic state against various antigens, including allogeneic cells or tissue transplants. These tolerogenic effects are mostly mediated through regulation of T cells such as inducing T cell anergy, T cell apoptosis, induction of Tregs, and affecting the local micro-environment toward a tolerogenic state by producing anti-inflammatory cytokines.

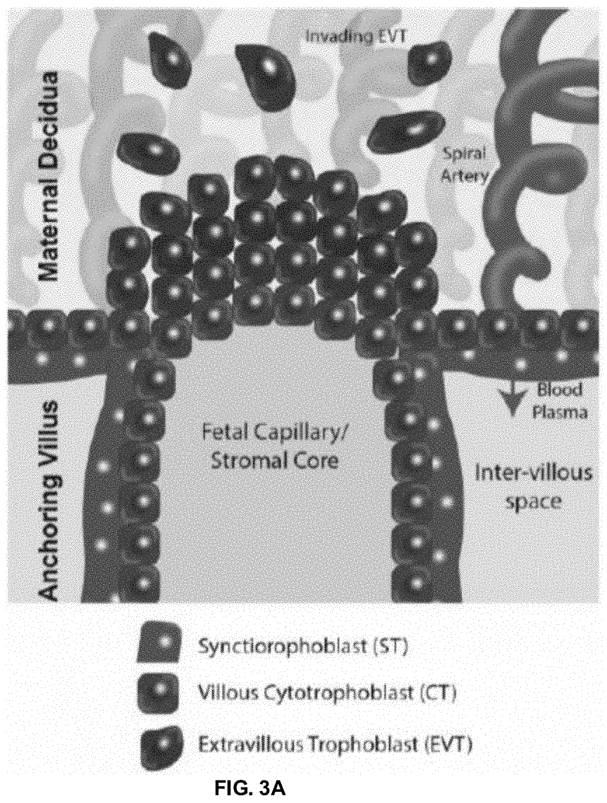

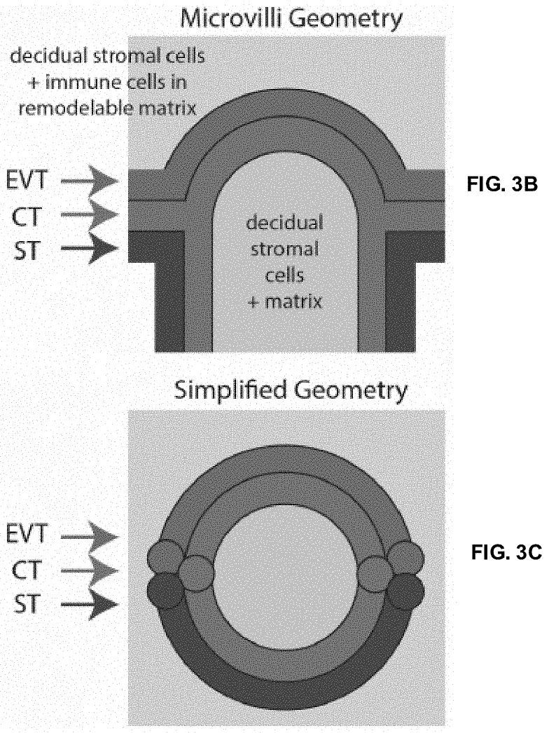

As will be appreciated by those of skill in the art, the live trophoblasts of the disclosed device can be selected based on the intended purpose of the device. Immunomodulatory trophoblasts fall into three general categories: cytotrophoblasts (CT), which are the proliferative progenitor cells to the other two subtypes—syncytiotrophoblasts (ST), which line the intervillous space, and extravillous trophoblasts (EVT), which contact with and migrate into the maternal decidua (FIG. 3A-3C).

In some aspects, the immunomodulatory trophoblasts are cytotrophoblasts (CT), syncytiotrophoblasts (ST), extravillous trophoblasts (EVT), or combinations thereof. These cells can be isolated from placenta using methods known in the art. In some aspects, the trophoblasts are trophoblast-like cell lines derived from trophoblasts or other cell types such as stem cells. In some aspects, trophoblast-like cell lines selected from JEG3, JAR, and combinations thereof.

In some aspects, the trophoblasts are organoids comprising trophoblasts. An organoid is a 3D multicellular in vitro tissue construct that mimics its corresponding in vivo organ, such that it can be used to replicate function of that organ in the tissue culture dish. It is thought that the processes that form these tissues in vitro approximate natural development or tissue maintenance.

### II. Vaccine Devices

Another aspect of the present disclosure encompasses an artificial placenta vaccine device. The device comprises trophoblasts; donor cells; and one or more macroencapsulation devices encapsulating the donor cells, the trophoblasts, or the donor cells and the trophoblasts. The donor cells can be from a tissue donor or are from the same source as the donor tissue.

The trophoblasts can be primary cells isolated from placenta. For instance, the trophoblasts are cytotrophoblasts (CT), syncytiotrophoblasts (ST), extravillous trophoblasts (EVT), or any combination thereof. The trophoblasts can also be human trophoblast-like cell lines selected from JEG3, JAR, and combinations thereof.

In some aspects, the trophoblasts are organoids comprising trophoblasts. The organoids comprise trophoblasts derived from a trophoblast-like cell line. In some aspects, the trophoblasts are organoids comprising trophoblasts, wherein the organoids are prepared using a method comprising the following steps: (a) providing or having provided trophoblasts; (b) embedding the trophoblasts in a degradable hydrogel comprising a 3D matrix and one or more adhesive ligands; and (c) incubating the trophoblasts under conditions and for a time sufficient for the trophoblasts to generate the organoids. The degradable hydrogel matrix can comprise maleimide-functionalized, 4-arm PEG cross-linked using protease-degradable peptides, or a PEG hydrogel crosslinked with a degradable crosslinker, a nondegradable crosslinker, or any combination thereof. The degradable crosslinker can be a protease-degradable peptides selected from VPM, half DTT-half VPM, GDQ, and GPQ-W, and any combination thereof. In some aspects, the degradable crosslinker is a VPM degradable peptide.

In some aspects, the macroencapsulation device is nondegradable, retrievable, or any combination thereof. In some aspects, the macroencapsulation device encapsulates the trophoblasts and the donor cells. In other aspects, the artificial placenta vaccine device comprises a first macroencapsulation device encapsulating the trophoblasts and a second macroencapsulation device encapsulating the donor cells, wherein a combination of the first macroencapsulation device and the second macroencapsulation device is operable to generate a vaccine when implanted into a subject in proximity to each other.

The macroencapsulation device can comprise vascular endothelial growth factor (VEGF). The macroencapsulation device can also comprise and adhesive peptide, such as but not limited to an RGD adhesive peptide.

In some aspects, the macroencapsulation device comprises a nondegradable biocompatible hydrogel matrix. The biocompatible hydrogel matrix can comprise multi-arm PEG functionalized with bioorthogonal reactive groups. In some aspects, the macroencapsulation device comprises a poly(ethylene glycol) (PEG) hydrogel matrix crosslinked using a nondegradable linker. The hydrogel matrix can be operable to be crosslinked within an injection mold, and wherein the hydrogel macroencapsulation device is formed from the injection mold. In some aspects, the hydrogel matrix comprises maleimide-functionalized, 4-arm PEG crosslinked using a nondegradable linker. In some aspects, the macroencapsulation device comprises a 20 kDa 4-arm PEG hydrogel matrix crosslinked using a nondegradable linker. In some aspects, the macroencapsulation device comprises a spiral geometry, a wrinkled sheet geometry, a planar sheet geometry, a branched geometry, or a vascular geometry.

In some aspects, nondegradable crosslinkers as contemplated herein include protease-degradable peptides selected from DTT, PEG-DT, and any combination thereof.

The hydrogel matrix can be functionalized with one or more adhesive peptides. The one or more adhesive peptide can be collagen I, collagen IV, laminin, fibronectin, fibrinogen, osteopontin, plasminogen, vitronectin, arginylglycylaspartic acid (RGD), or any combination thereof. The one or more adhesive peptide is collagen IV, laminin, arginylglycylaspartic acid (RGD), or any fraction thereof, or any combination thereof. In some aspects, the adhesive peptide is carginylglycylaspartic acid (RGD). The one or more adhesive peptides can be 3, 4, 5, 6, 7, 8, 9 or 10 amino acids in length. The one or more adhesive peptide can comprise an amino acid sequence selected from RGD, RDG, GFOGER, YIGSR, IKVAV, REDV, DGEA, VGVAPG, GRGDS, LDV, RGDV, PDSGR, RYVVLPR, LGTIPG, LAG, RGDS, RGDF, HHLGGALQAGDV, VTCG, SDGD, GREDVY, GRGDY, GRGDSP, VAPG, GGGGRGDSP, GGGGRGDY, FTLCFD, or any combination thereof.

In some aspects, the artificial placenta vaccine device comprises donor cells; organoids comprising trophoblasts, and one or more hydrogel matrix encapsulating the donor cells and organoids, the organoids, or the donor cells, the organoids. The hydrogel matrix comprises a 3D matrix structure comprising a crosslinked poly(ethylene glycol) (PEG) hydrogel comprising maleimide-functionalized, 20 kDa 4-arm PEG cross-linked using a nondegradable peptide selected from DTT, PEG-DT, and any combination thereof; RGD adhesive peptide; and vascular endothelial growth factor (VEGF).

In some aspects, the artificial placenta vaccine device comprises organoids comprising trophoblasts and allogeneic cells, and one or more macroencapsulation devices encapsulating the donor cells, the organoids, or the donor cells and the organoids. The organoids are prepared using a method comprising: (a) providing or having provided trophoblasts; (b) embedding the trophoblasts in a degradable hydrogel comprising a 3D matrix and one or more adhesive ligands; and (c) incubating the cells under conditions and for a time sufficient for the trophoblasts to generate organoids comprising the trophoblasts. The degradable hydrogel matrix can consist of or comprise maleimide-functionalized, 4-arm PEG cross-linked using protease-degradable peptides. In some aspects, the hydrogel matrix comprises a PEG hydrogel crosslinked with degradable crosslinkers, nondegradable crosslinker, or any combination thereof.

Degradable crosslinkers include but are not limited to protease-degradable peptides selected from VPM, half DTT-half VPM, GDQ, and GPQ-W, and any combination thereof. In some aspects, the degradable crosslinker is a VPM protease-degradable peptide.

### III. Mimic Devices

Yet another aspect of the present disclosure encompasses a placenta-mimicking device. The placenta-mimicking device comprises allogeneic cells, trophoblasts, and one or more macroencapsulation devices encapsulating the allogeneic cells, the trophoblasts, or the allogeneic cells and the trophoblasts.

In some aspects, the allogeneic cells are therapeutic cells. As used herein, the term “allogeneic” when applied to cells or tissues of the instant disclosure refers to any cell or tissue that, when transplanted into a subject, induces an immune response against the cells. In some aspects, the allogeneic cells can be a therapeutic cell population. Therapeutic cells for cell therapy. Cell therapy (also called cellular therapy, cell transplantation, or cytotherapy) is a therapy in which viable cells are injected, grafted or implanted into a patient in order to effectuate a medicinal effect, for example, by transplanting T-cells capable of fighting cancer cells via cell-mediated immunity in the course of immunotherapy, or cells that have the capacity to release soluble factors such as cytokines, chemokines, and growth factors which act in a paracrine or endocrine manner. In some aspects, the cells are cells capable of producing insulin.

The trophoblasts can be primary cells isolated from placenta. For instance, the trophoblasts are cytotrophoblasts (CT), syncytiotrophoblasts (ST), extravillous trophoblasts (EVT), or any combination thereof. The trophoblasts can also be human trophoblast-like cell lines selected from JEG3, JAR, and combinations thereof.

In some aspects, the trophoblasts are organoids comprising trophoblasts. The organoids comprise trophoblasts derived from a trophoblast-like cell line. In some aspects, the trophoblasts are organoids comprising trophoblasts, wherein the organoids are prepared using a method comprising the following steps: (a) providing or having provided trophoblasts; (b) embedding the trophoblasts in a degradable hydrogel comprising a 3D matrix and one or more adhesive ligands; and (c) incubating the trophoblasts under conditions and for a time sufficient for the trophoblasts to generate the organoids. The degradable hydrogel matrix can comprise maleimide-functionalized, 4-arm PEG cross-linked using protease-degradable peptides, or a PEG hydrogel crosslinked with a degradable crosslinker, a nondegradable crosslinker, or any combination thereof. The degradable crosslinker can be a protease-degradable peptides selected from VPM, half DTT-half VPM, GDQ, and GPQ-W, and any combination thereof. In some aspects, the degradable crosslinker is a VPM degradable peptide.

In some aspects, the macroencapsulation device is nondegradable, retrievable, or any combination thereof. In some aspects, the macroencapsulation device encapsulates the trophoblasts and the allogeneic cells. In other aspects, the placenta-mimicking device comprises a first macroencapsulation device encapsulating the trophoblasts and a second macroencapsulation device encapsulating the allogeneic cells, wherein a combination of the first macroencapsulation device and the second macroencapsulation device is operable to generate a vaccine when implanted into a subject in proximity to each other.

The macroencapsulation device can comprise vascular endothelial growth factor (VEGF). The macroencapsulation device can also comprise RGD adhesive peptide.

In some aspects, the macroencapsulation device comprises a nondegradable biocompatible hydrogel matrix. The biocompatible hydrogel matrix can comprise multi-arm PEG functionalized with bioorthogonal reactive groups. In some aspects, the macroencapsulation device comprises a poly(ethylene glycol) (PEG) hydrogel matrix crosslinked using a nondegradable linker. The hydrogel matrix can be operable to be crosslinked within an injection mold, and wherein the hydrogel macroencapsulation device is formed from the injection mold. In some aspects, the hydrogel matrix comprises maleimide-functionalized, 4-arm PEG crosslinked using a nondegradable linker. In some aspects, the macroencapsulation device comprises a 20 kDa 4-arm PEG hydrogel matrix crosslinked using a nondegradable linker. In some aspects, the macroencapsulation device comprises a spiral geometry, a wrinkled sheet geometry, a planar sheet geometry, a branched geometry, or a vascular geometry.

In some aspects, the nondegradable crosslinkers are protease-degradable peptides selected from DTT, PEG-DT, and any combination thereof.

The hydrogel matrix can be functionalized with one or more adhesive peptides. The one or more adhesive peptide can be collagen I, collagen IV, laminin, fibronectin, fibrinogen, osteopontin, plasminogen, vitronectin, arginylglycylaspartic acid (RGD), or any combination thereof. The one or more adhesive peptide is collagen IV, laminin, arginylglycylaspartic acid (RGD), or any fraction thereof, or any combination thereof. In some aspects, the adhesive peptide is carginylglycylaspartic acid (RGD). The one or more adhesive peptides can be 3, 4, 5, 6, 7, 8, 9 or 10 amino acids in length. The one or more adhesive peptide can comprise an amino acid sequence selected from RGD, RDG, GFOGER, YIGSR, IKVAV, REDV, DGEA, VGVAPG, GRGDS, LDV, RGDV, PDSGR, RYVVLPR, LGTIPG, LAG, RGDS, RGDF, HHLGGALQAGDV, VTCG, SDGD, GREDVY, GRGDY, GRGDSP, VAPG, GGGGRGDSP, GGGGRGDY, FTLCFD, or any combination thereof.

In some aspects, the placenta-mimicking device comprises cells capable of producing insulin, organoids comprising trophoblasts, and one or more hydrogel matrix encapsulating the cells capable of producing insulin and organoids, the organoids, or the cells capable of producing insulin, the organoids. The hydrogel matrix comprises a 3D matrix structure comprising a crosslinked poly(ethylene glycol) (PEG) hydrogel comprising maleimide-functionalized, 20 kDa 4-arm PEG cross-linked using a nondegradable peptide selected from DTT, PEG-DT, and any combination thereof; RGD adhesive peptide; and vascular endothelial growth factor (VEGF). In some aspects, the cells capable of producing insulin produce insulin in a glucose-dependent manner.

In some aspects, the placenta-mimicking device comprises organoids comprising trophoblasts and allogeneic cells, and one or more macroencapsulation devices encapsulating the allogeneic cells, the organoids, or the allogeneic cells and the organoids. The organoids are prepared using a method comprising: (a) providing or having provided trophoblasts; (b) embedding the trophoblasts in a degradable hydrogel comprising a 3D matrix and one or more adhesive ligands; and (c) incubating the cells under conditions and for a time sufficient for the trophoblasts to generate organoids comprising the trophoblasts. The degradable hydrogel matrix can comprise maleimide-functionalized, 4-arm PEG cross-linked using protease-degradable peptides. In some aspects, the hydrogel matrix comprises a PEG hydrogel crosslinked with degradable crosslinkers, nondegradable crosslinker, or any combination thereof.

The degradable crosslinkers can be protease-degradable peptides selected from VPM, half DTT-half VPM, GDQ, and GPQ-W, and any combination thereof. In some aspects, the degradable crosslinker is a VPM protease-degradable peptide.

### IV. Method of Preparing Organoids

Another aspect of the present disclosure encompasses a method of preparing organoids comprising trophoblasts. Methods of preparing organoids are known in the art, and currently use off-the-shelf, one-size-fits-all hydrogels that are not suitable for optimal growth of trophoblast organoids. However, the inventors surprisingly discovered a degradable synthetic, modular hydrogel can be adapted to tailor to the generation of trophoblast organoids. The hydrogels can be adapted by using various adhesive ligands and crosslinkers to establish what is optimal to generate the organoids. In general, organoids are grown in degradable, supportive, 3D matrices for islet culture and transplantation were previously optimized, and this approach is used to adapt the hydrogel matrix composition to mimic the trophoblast microenvironment. (See, e.g., Weaver, J D, et al., Science Advances, 2017. 3(6): p. e1700184, the disclosure of which is incorporated herein in its entirety). Suitable hydrogels, adhesive ligands and crosslinkers for preparing optimal trophoblast hydrogels can be as described in Section I(a) herein above.

The method comprises providing or having provided trophoblasts; embedding the trophoblasts in a degradable hydrogel comprising a 3D matrix and one or more adhesive ligands; and incubating the cells under conditions and for a time sufficient for the trophoblasts to generate organoids comprising the trophoblasts. The hydrogel matrix can, by way of non-limiting example, comprise maleimide-functionalized, 4-arm PEG cross-linked using protease-degradable peptides.

Organoids can be derived from primary trophoblasts, stem cells, from trophoblast-like cell lines selected from JEG3, JAR, and combinations thereof. In some aspects, organoids of the instant disclosure are derived from primary trophoblasts. In other aspects, organoids of the instant disclosure are derived from trophoblast-like cell lines such as JEG3, JAR, and combinations thereof. In yet other aspects, organoids of the instant disclosure are derived from stem cells.

In some aspects, the hydrogel matrix can comprise one or more peptides and/or bioactive signals. In an aspect, one or more of the peptides and/or bioactive signals can be bonded to one or more components of the hydrogel matrix. In an aspect, one or more of the peptides and/or bioactive signals can be irreversibly bonded to one or more components of the hydrogel matrix. In an aspect, one or more of the peptides and/or bioactive signals can be reversibly bonded to one or more components of the hydrogel matrix. In some aspects, one or more of the peptides and/or bioactive signals can be soluble in the hydrogel matrix. The one or more of the peptides and/or bioactive signals can be added to the hydrogel matrix before crosslinking or after crosslinking. The degradable crosslinkers can be protease-degradable peptides selected from VPM, half DTT-half VPM, GDQ, and GPQ-W, and any combination thereof. In some aspects, the degradable crosslinker is a VPM protease-degradable peptide.

The hydrogel matrix can comprise PEG, agarose, alginate, or any combination thereof. In some aspects, the hydrogel matrix comprises alginate.

In other aspects, the hydrogel matrix comprises maleimide-functionalized, 4-arm PEG cross-linked using protease-degradable peptides. In some aspects, the degradable hydrogel matrix comprises a PEG hydrogel crosslinked with degradable crosslinkers, nondegradable crosslinker, or any combination thereof. The degradable crosslinkers are protease-degradable peptides selected from VPM, GDQ, GDQ-W, and any combination thereof. In one aspect, the degradable crosslinker is a VPM degradable peptide.

### V. Methods of Immunosuppression Free Transplantation

Yet another aspect of the present disclosure encompasses a method of preventing and/or treating an immunological response to a transplanted tissue in a subject in need thereof. Without wishing to be bound by any one theory, it is believed that implantation of a placenta vaccine device comprising encapsulated trophoblasts and cells derived from the donor allograft will synergize to generate tolerance against donor antigens. Donor antigens released from the transplanted tissue and the donor cells in the device in the presence of trophoblast-secreted tolerogenic factors will induce a tolerogenic immune phenotype toward donor antigens, as in pregnancy.

The method comprises implanting an artificial placenta vaccine device; and transplanting donor tissue into the subject concurrently with or prior to implanting the artificial placenta vaccine device. The artificial placenta vaccine device can be as described in Section II herein above.

The macroencapsulation device can encapsulate the trophoblasts and the donor cells. Alternatively, the placenta-mimicking device can comprise a first macroencapsulation device encapsulating the trophoblasts and a second macroencapsulation device encapsulating the donor cells, and the method comprises implanting the first macroencapsulation device and the second macroencapsulation device.

The device is implanted in the subject at a period of time relative to the time of transplantation of donor material sufficiently long for tolerance toward the donor material is established, enabling transplant of allogeneic material in the absence of chronic systemic immunosuppression. The time at which the device is implanted in the subject relative to the time of transplantation of donor tissue can and will vary depending on the donor tissue, the device, the immunological response to the donor tissue, among other variables and can be determined experimentally using methods known in the art. The device can be implanted prior to tissue transplant or at the time of transplant. During this time, tolerance toward the donor cells is established, enabling transplant of allogeneic cells/tissue/organs in the absence of chronic systemic immunosuppression. In some aspects, the device is implanted simultaneously with the transplanted tissue. In other aspects, the device is implanted about 1-30 days after transplant.

The device is implanted in the subject at a location in the subject appropriate for tolerance to the donor material is established, enabling transplant of allogeneic tissue or cells in the absence of chronic systemic immunosuppression. Generally, the implant location also provides an appropriate environment for survival of the trophoblasts and donor cells in the artificial placenta vaccine device. Such an environment can provide optimal oxygenation and nutrient exchange for a functioning device. Additionally, the location at which the device is implanted in subject can and will vary depending on the donor tissue, the device, the immunological response to the donor tissue, among other variables and can be determined experimentally using methods known in the art. The device can be implanted in proximity to the transplant site. In some aspects, the device is implanted in the omentum. In other aspects, the device is implanted subcutaneously. The device can be implanted in the omentum or subcutaneously and can be implanted in proximity to each other.

In some aspects, the method comprises implanting an artificial placenta vaccine device into the subject; and transplanting donor tissue into the subject concurrently with or prior to implanting the artificial placenta vaccine device.

One aspect of the instant disclosure encompasses an immunosuppression-free tissue transplantation method. The method implanting an artificial placenta vaccine device into the subject; and transplanting donor tissue into the subject concurrently with or prior to implanting the artificial placenta vaccine device.

One aspect of the instant disclosure encompasses the use of one or more artificial placenta vaccine device for transplanting donor tissue into a subject in need thereof in the absence of chronic systemic immunosuppression.

### VI. Methods of Using Devices Comprising Allogeneic Cells

An additional aspect of the present disclosure encompasses a method of preventing and/or treating an immunological response to transplanted allogeneic cells in a subject in need thereof. Typical immuno-engineering approaches to induce tolerance include methods targeting specific antigens or immunological pathways, or localized delivery or presentation of tolerogenic factors. These methods are highly simplistic, delivering a single antigen or activating a single pathway, and involve transient communication with the immune system, which necessitates replenishment or replacement over time to maintain tolerance. In the context of pancreatic islet cell transplant, local delivery of an immunomodulatory signal within the islet graft site has been explored, but tolerance induction still required short-term low-dose administration of systemic immunosuppressive agents.

Without wishing to be bound by any one theory, it is believed that implantation of a placenta vaccine device comprising encapsulated trophoblasts and cells derived from the donor allograft will synergize to generate tolerance against donor antigens. Donor antigens released from the transplanted tissue and the donor cells in the device in the presence of trophoblast-secreted tolerogenic factors will induce a tolerogenic immune phenotype toward donor antigens, as in pregnancy. Without wishing to be bound by any one theory, it is believed that implantation of a placenta vaccine device comprising encapsulated trophoblasts and cells derived from the donor allograft will synergize to generate tolerance against donor antigens. Donor antigens released from the transplanted tissue and the donor cells in the device in the presence of trophoblast-secreted tolerogenic factors will induce a tolerogenic immune phenotype toward donor antigens, as in pregnancy.

The method comprises implanting a placenta-mimicking device comprising allogeneic cells; trophoblasts; and one or more macroencapsulation devices encapsulating the allogeneic cells, the trophoblasts, or the allogeneic cells and the trophoblasts. In some aspects, the macroencapsulation device encapsulates the trophoblasts and the allogeneic cells. In other aspects, the placenta-mimicking device comprises a first macroencapsulation device encapsulating the trophoblasts and a second macroencapsulation device encapsulating the allogeneic cells, and the method comprises implanting the first macroencapsulation device and the second macroencapsulation device. In one aspect, the first encapsulation device is implanted between about 1-30 days prior to implanting the second encapsulation device. The first and the second encapsulation devices are implanted in proximity to each other.

Generally, the implant location also provides an appropriate environment for survival of the trophoblasts and donor cells in the artificial placenta vaccine device. Such an environment can provide optimal oxygenation and nutrient exchange for a functioning device. In some aspects, the device is implanted in the omentum or subcutaneously.

Another aspect of the present disclosure encompasses a method of treating diabetes Type I in a subject in need thereof. The method comprises implanting a placenta-mimicking device comprising cells capable of producing insulin; trophoblasts; and one or more macroencapsulation devices encapsulating the cells capable of producing insulin, the trophoblasts, or the allogeneic cells and the trophoblasts. In some aspects, the cells capable of producing insulin produce insulin in a glucose-dependent manner. In some aspects, the cells capable of producing insulin are selected from the group consisting of differentiated stem cells along the pancreatic lineage, cells transfected with an glucose-inducible insulin promoter, pancreatic islet cells, pancreatic progenitor cells, ductal pancreatic progenitor cells, and islets or islet cells. In some aspects, the cells capable of producing insulin are islets or islet cells.

### VII. Kits

A further aspect of the present disclosure encompasses kits comprising one or more artificial placenta vaccine devices for use in a method of method of preventing and/or treating an immunological response to a transplanted tissue in a subject in need thereof, a method of immunosuppression-free tissue transplantation, or combinations thereof. Artificial placenta vaccine devices can be as described in Sections I, and II, and use in a method of immunosuppression-free tissue transplantation can be as described in Section V.

The kits can also comprise one or more placenta-mimicking devices for use in a method of preventing and/or treating an immunological response to transplanted allogeneic cells, a method of treating diabetes Type I in a subject in need thereof, and combinations thereof. Artificial placenta vaccine devices can be as described in Sections I, and III, and use in a method of immunosuppression-free tissue transplantation can be as described in Section VI.

A kit can comprise one or more components for making or using any of the devices described herein. Other aspects of this disclosure include kits containing all or a portion of the components of the disclosed devices.

Kits according to the present disclosure may include one or more additional reagents useful for practicing the methods according to the present disclosure. A kit generally includes a package with one or more containers holding the reagents, as one or more separate compositions or, optionally, as admixture where the compatibility of the reagents will allow. The test kit can also include other material(s), which may be desirable from a user standpoint, such as a buffer(s), a diluent(s), a standard(s), and/or any other material useful in processing or conducting any other step of the tagging method.

Kits according to the present disclosure preferably include instructions for preparing and using devices of the instant disclosure. Instructions included in kits of the present disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

It should be understood from the foregoing that, while particular aspects have been illustrated and described, various modifications can be made thereto without departing from the spirit and scope of the invention as will be apparent to those skilled in the art. Such changes and modifications are within the scope and teachings of this invention as defined in the claims appended hereto.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there can be additional elements other than the listed elements.

As various changes could be made in the above-described cells and methods without departing from the scope of the invention, it is intended that all matter contained in the above description and in the examples given below, shall be interpreted as illustrative and not in a limiting sense.

### Examples

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Engineering Translational Hydrogel Macroencapsulation Devices

There is substantial risk associated with the chronic use of immunosuppression in organ and cell transplantation. As a result, research has explored the development of devices to isolate transplanted cells from the recipient immune system. Device designs with the greatest potential for translation to the clinic include macroencapsulation devices, cell encapsulation devices which prioritize whole graft containment, retrievability, and safety. To date, these devices have demonstrated limited pre-clinical and clinical efficacy, due in large part to device transport issues: the diffusion of sufficient oxygen within the device to support encapsulated cell survival, as well as efficient insulin transport out of the device. Therefore, a critical need exists for rationally designed macroencapsulation devices with optimal geometry that minimizes diffusion distances in order to maximize graft survival and function. The use of vasculogenic degradable hydrogels to enhance vascularization, and therefore oxygenation, at the surface of macroencapsulation devices has been demonstrated. Despite improved vascularization, non-ideal device geometry limits encapsulated cell viability and function in vivo, with in silico finite element modeling of device oxygenation indicating non-optimal spatial distribution of oxygen within the device. As such, the goal of this study is to design a clinically translatable macroencapsulation device that maximizes islet viability and function through optimization of geometry and encapsulating material, and which prioritizes facile device implementation in the clinic. Three different types of encapsulation are studied. These are shown in FIG. 6B-D.

Injection molding can be used to generate complex 3D hydrogel device geometries. To generate complex 3D hydrogel macroencapsulation device geometries in a manner that is translatable and able to be implemented with minimal training in the clinic, individual components (hydrogel components, injection mold), can be provided as an off-the=shelf product. 3D printing can be used to rapidly iterate and prototype injection mold designs (FIG. 6A). To generate hydrogels in an injection molding strategy, a synthetic hydrogel crosslinking reaction with kinetics that allow sufficient time to inject components into the mold prior to gelation is necessary. A biorthogonal reaction library with a range of kinetics to select ideal candidates for macroencapsulation device fabrication will be developed (see FIG. 6F).

### Example 2. Development of Synthetic Hydrogel-Based Artificial Placenta to Probe Trophoblast Immunomodulatory Mechanisms

In order to use trophoblasts as a cell-based therapy to induce tolerance toward allogeneic cells and tissues, first an artificial niche to support trophoblast viability and function, and to promote and maintain the most tolerogenic phenotype will be engineered. Three-dimensional (3D) synthetic hydrogel matrices will be used to generate and define this artificial niche11, and this artificial placenta system will be used to investigate the immunomodulatory capacity of encapsulated trophoblasts.

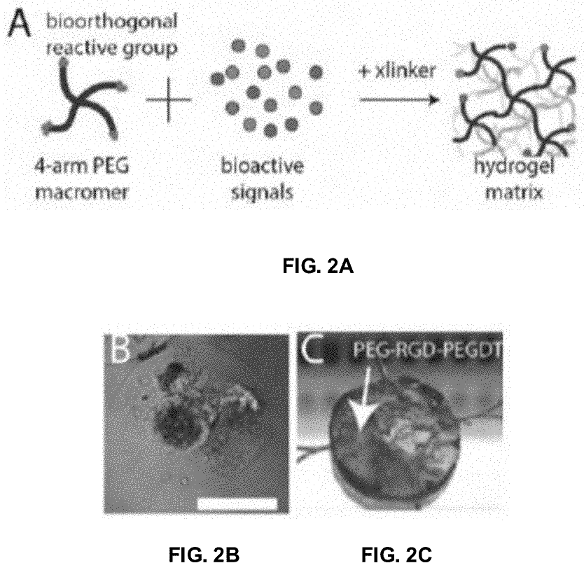

3D hydrogel matrices can serve as artificial niches to investigate and drive cell differentiation and behavior. By incorporating cues in a synthetic hydrogel matrix, as well as through soluble signaling, the behavior of encapsulated cells11 can be driven. 3D synthetic hydrogel matrices (FIG. 2A) will be used to develop an in vitro artificial placenta. Synthetic hydrogel platforms can be used for a wide range of applications (FIG. 2B, C). This synthetic poly(ethylene glycol)-based hydrogel system is tunable, allowing incorporation of peptides such as adhesive ligand RGD, as well as bioactive compounds such as vascular endothelial growth factor (VEGF). We have previously optimized degradable and nondegradable, supportive, 3D matrices for islet culture and transplantation, and aim to use this approach to adapt the hydrogel matrix composition to mimic the trophoblast microenvironment. There is a need to develop a well-defined, synthetic hydrogel-based method for controlled trophoblast culture and differentiation.

The influence of both matrix-bound and soluble cues on the long-term survival, differentiation, and function of trophoblasts within 3D hydrogel matrices are investigated. First, the influence of adhesive peptides derived from extracellular matrix components that dominate the placenta, including collagen IV and laminin are examined. Then, known soluble cues for influencing trophoblast differentiation are examined. Last, the influence of trophoblast cell subtype, composition, and architecture on the tolerogenic capacity of synthetic hydrogel-based artificial placenta constructs is examined.

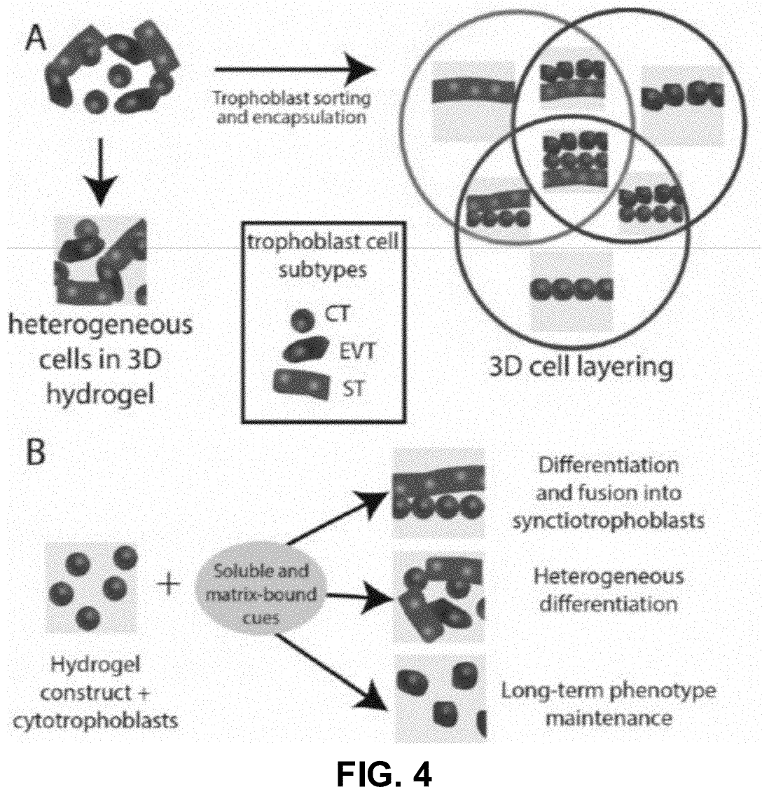

The native placenta architecture consists of branching villi chiefly composed of trophoblasts, which fall into three general categories: cytotrophoblasts (CT), which are the proliferative progenitor to the other two subtypes—syncytiotrophoblasts (ST), which line the intervillous space, and extravillous trophoblasts (EVT), which contact with and migrate into the maternal decidua (FIG. 3A). All three cell phenotypes have been documented to express or secrete various immunomodulatory molecules, but it is unclear whether all three must act synergistically to establish and maintain tolerance. This is investigated through two main pathways: (1) the influence of cell subtype on tolerogenic capacity (FIG. 4A); and (2) the influence of matrix-bound cues, including adhesion molecules and matrix metalloproteinase (MMP) degradable peptide crosslinker kinetics on the differentiation and reorganization of trophoblasts within a three dimensional hydrogel matrix (FIG. 4B).

Tissue architecture and cell subtype contribution to placenta immunomodulation is determined by trophoblast cell sorting into the three primary subtypes, and encapsulation in optimized synthetic hydrogels according to the combinations demonstrated in FIG. 4A. Sequential layering of cells embedded within synthetic hydrogels is used to generate 3D structures that mimic the native placental villi architecture. The trophoblast secretome of these constructs are sampled over time in culture and analyzed by Luminex multiplex assays to determine the temporal secretion patterns of trophoblasts within a 3D synthetic hydrogel matrix under the proposed cell subtype combinations and architectures. Additionally, as CTs are precursors to STs and EVTs, it is investigated whether stable immunomodulatory trophoblast structures are derived from encapsulation and differentiation of CTs or CT organoids within synthetic hydrogel matrices (FIG. 4B). For example, in the native placenta, CTs maintain a near-constant 1:9 ratio to STs throughout pregnancy; therefore, the influence of soluble and synthetic matrix-bound cues on CT differentiation and phenotype maintenance, and the resulting tolerogenic features of these structures are investigated.

Following characterization of the influence of hydrogel conditions on trophoblasts, optimal tolerogenic candidates are selected to investigate the direct influence of the artificial placenta on immune cells in vitro. First phase studies investigate primary mouse trophoblast influence on innate immune cells, including APCs such as macrophages and DCs. Second phase mechanistic studies investigate the influence of primary mouse trophoblast secretome on OVA peptide antigen-specific OT-I (CD8) and OT-II (CD4) T cell priming and activation in the presence of APCs. T cell phenotype and proliferation after coculture with plated or encapsulated trophoblasts are investigated, either in the presence of soluble OVA in the medium, or co-encapsulated trophoblasts and the OVA-secreting EG-7 cell line.

Multiple additional factors are considered in the generation of an artificial placenta-mimicking implant, including: (1) the influence of gestational time point on trophoblast immunomodulatory potency, which has been shown to fluctuate over the course of gestation; (2) the influence of tissue oxygenation on immunomodulatory capacity, as trophoblast oxygenation levels vary greatly between the first and second trimesters; and (3) last, this system of EG-7 and trophoblast co-encapsulation is used to elucidate the necessary ratio of trophoblast (and therefore soluble tolerogenic factors) to antigen-secreting cell to maximize tolerogenic effect.

As this study aims to translate these constructs to a murine model in vivo, murine immune tools are used to investigate immunomodulatory mechanisms of murine trophoblasts. Trophoblast immunology and placenta extracellular matrix composition can vary significantly between species, and mechanisms which can exist in rodents can not translate to human immunology. As such, (1) human trophoblast cell lines (JEG3, JAR), and (2) primary human trophoblasts from deidentified human placenta samples, purchased commercially or obtained from gestational tissue banks, are used to evaluate and confirm significant mechanisms of human trophoblast immune evasion. Human primary trophoblasts and cell lines in the described synthetic hydrogel matrices are reevaluated to ensure comparable function. Human trophoblasts with human T cells in the artificial placenta—T cell coculture model to ensure comparable trends in secreted factors and influence on activated immune cells are evaluated. These additional studies facilitate downstream translatability of this approach.

### Example 3. Placental Mimicry for Immunosuppression-Free Cell Transplantation

Tolerogenic artificial placenta constructs developed in Example 2 are used to induce tolerance toward allogeneic islet cell grafts in a murine model of chemically-induced diabetes. In islet transplantation, a cell-based therapy to replace malfunctioning insulin-producing cells in type 1 diabetic patients, the risks associated with chronic systemic immunosuppression outweigh the risks of long-term diabetes-associated morbidities. As such, islet transplantation is precluded in the wider type 1 diabetes population until a method of immunosuppression-free islet transplantation can be devised.

Islet encapsulation within a biomaterial has long been proposed as a means to reduce the immune response to transplanted grafts, whereby a physical barrier on the islet surface prevents direct antigen recognition by immune cells. This can be described as analogous to one mechanism of trophoblast immune evasion, wherein they present an immunologically inert barrier at the fetal-maternal interface. Traditional encapsulation techniques have used hydrogel microcapsules, on the scale of 600-1000 μm diameter, with limited translational success due to safety limitations of non-retrievable capsules delivered within the intraperitoneal space, where human trials have demonstrated microcapsule adhesion to parietal peritoneum, spleen, kidney, and omentum. As such, macroencapsulation devices for islet encapsulation have been explored in preclinical and clinical trials. While they confer the safety benefit of a single, retrievable device, functional success of macroencapsulation devices has been limited, due in large part to both oxygen availability to encapsulated islets and immune response to the graft. Thus, strategically designed macroencapsulation devices that minimize oxygen diffusion distances and incorporate synergistic immunomodulatory approaches are needed to realize fully functional and immunoprotected islet grafts.

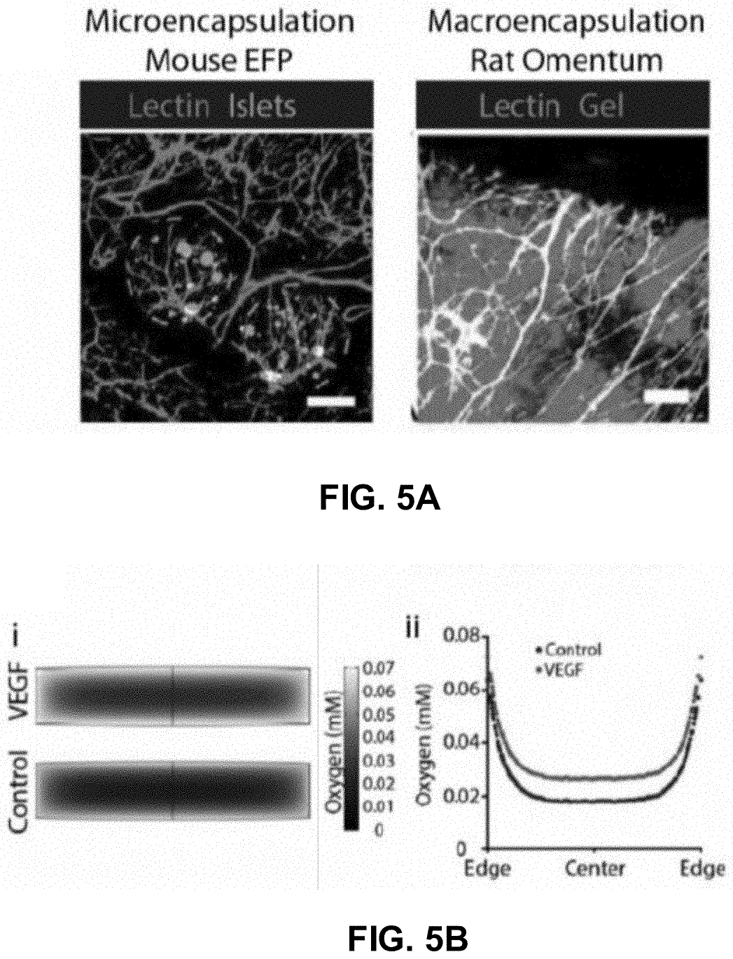

Synthetic PEG-based hydrogels was used to encapsulate and transplant islets in both microencapsulation (FIG. 2B) and macroencapsulation (FIG. 2C) conformations. A vasculogenic, degradable hydrogel system was used to deliver encapsulated islets to the isolated, retrievable, and highly vascularized omentum or the murine equivalent, the epididymal fat pad (EFP). This vasculogenic hydrogel remodels over a few weeks, promoting vascularization at the device surface (FIG. 5A). While microencapsulated islets thrived in the murine EFP, macroencapsulated islets experienced suboptimal oxygenation in the rat omentum (FIG. 5B) and failed to reverse diabetes, despite enhanced vascularization at the device surface. These studies highlight the impact of oxygen gradients within macroencapsulation devices on cell survival and function.

To address these issues, a method to injection mold complex 3D hydrogel structures for islet macroencapsulation (FIG. 6A) was developed. Finite element modeling was used to optimize macroencapsulation device geometry for maximal oxygenation of encapsulated cells (FIG. 6C). Injection molds were 3D printed, allowing rapid prototyping of macroencapsulation device designs. Injection molding was selected as the mode of hydrogel fabrication for its translatability, as the mold and hydrogel components can be provided as an off-the-shelf kit and implemented in the clinic by a minimally trained clinician. This versatile system can readily accommodate either homogeneous co-encapsulation of cells, or compartmentalized encapsulation depending on the optimal conformation determined in Example 2 (FIG. 6E). It is expected that ideal hydrogel composition for the artificial placenta will differ significantly from that required for islet encapsulation. With encapsulated islets, synthetic hydrogels are designed to restrain the diffusion of molecules larger than insulin (˜7 kDa), whereas in the artificial placenta, the free diffusion of trophoblast-secreted tolerogenic particles as large as exosomes are desired. A fabrication method enabling a multi-component hydrogel design, such as injection molding, can be needed for the artificial placenta.

In this study, injection molded hydrogel macroencapsulation devices are used to deliver co-encapsulated allogeneic islets and trophoblasts to the EFP in a chemically-induced diabetic mouse model. Encapsulation serves to (1) house delivered cells in a defined and retrievable device, and (2) eliminate direct antigen recognition of islet tissue. Thus, islet antigen recognition is restricted to indirect antigen recognition, which it is anticipated will be thwarted by trophoblast-secreted tolerogenic factors within the graft microenvironment and the draining lymph node. The impact of haplotype mismatch between transplant recipient (C57BL6J [H2 b]), islet donor (Balb/c [H2 d]), and trophoblast donor (C57BL/6J, Balb/c, or C3H [H2 k]) on graft function and recipient immune response are examined. An efficacious third-party (C3H) trophoblast strategy would be the most translatable configuration, as primary trophoblast donors can be major histocompatibility complex (MHC) mismatched from islet donors in a clinical setting. Graft recipients are monitored for length of diabetes reversal against unmodified and encapsulated control islet grafts. Longer-term reversal of trophoblast-containing grafts, and a potentially reduced islet requirement for diabetes reversal due to trophoblast-secreted anti-inflammatory factors is anticipated. At terminal endpoints of in vivo experiments, grafts are removed to demonstrate recipient return to hyperglycemia, and to confirm graft-dependent diabetes reversal. While long-term diabetes reversal demonstrates graft function, it lacks the sensitivity to quantify graft viability in real time. As such, longitudinal non-invasive in vivo imaging of luciferase-expressing islets are used to investigate allogeneic cell survival quantitatively in real time. To investigate immune responses to the graft, excised grafts and draining lymph nodes are evaluated histologically and via flow cytometry for immune cell populations of interest.

At the onset of pregnancy, 40% of maternal decidual cells are leukocytes, primarily natural killer cells, T cells, dendritic cells, and macrophages; additionally, the uterine draining lymph node plays a large role in educating T regulatory cells towards a tolerogenic phenotype 1. Therefore, flow cytometry and histological analysis is also used to investigate early immunological responses within the graft site and draining lymph node. Once graft function is evaluated in a chemically induced diabetic mouse model, the impact of this strategy in an autoimmune model of diabetes, the non-obese diabetic mouse, are explored to evaluate whether antigen-specific tolerance can be induced after established autoimmunity.

An additional factor that can influence trophoblast survival in vivo is species specific gestational lengths. We will investigate mouse and human trophoblast long-term survival in vivo, and whether trophoblast survival can be lengthened by incorporating matrix-bound cues reminiscent of early placenta extracellular matrix composition. As such, we anticipate that encapsulated human trophoblasts could be engineered for in vivo viability up to at least one year. Last, evidence of long-term chimerism between mother and fetus, wherein fetal cells persist in the mother for years post parturition, indicates that tolerance could persist long-term post trophoblast death.

Another consideration for this system is the influence of recipient biological sex on trophoblast survival and ability to induce tolerance. The hormones progesterone and estrogen play key roles in the regulation of pregnancy, and trophoblasts themselves, and it is unclear how allogeneic trophoblasts would respond to the male hormone milieu, or how trophoblast implants would impact male or female recipient hormonal regulation or overall physiology. We will evaluate the impact of sex in both donor trophoblast cells and recipients on the efficacy of this strategy, and their impact on recipient physiology.

Last, we can simplify our model to a luciferase-expressing encapsulated OVA secreting cell line (EG-7) monitored longitudinally and quantitatively for viability via noninvasive in vivo imaging in the OVA-specific OT-I and OT-II mouse models. This approach will enable the mechanistic investigation of an antigen-specific immune response prior to implementation in a more complex cell transplantation model.

### Example 4. Performance of Trophoblast-Like Cell Lines in PEG Gets with Varying Crosslinkers and Bioactive Signals

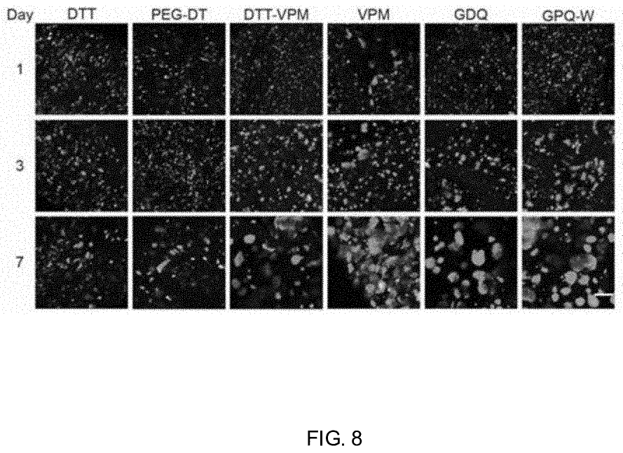

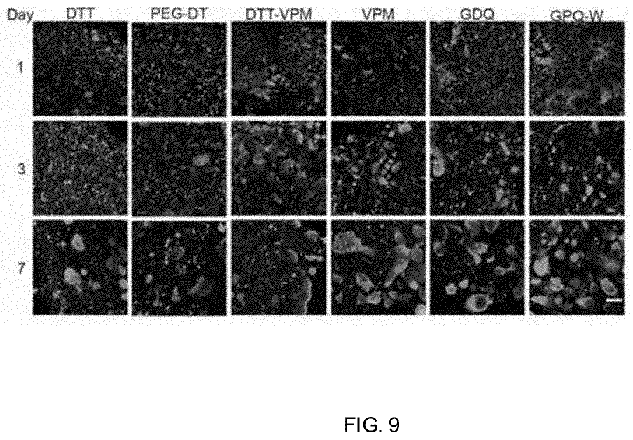

The performance of JEG-3 and JAR trophoblast-like cell lines was evaluated in PEG gels with varying crosslinkers and bioactive signals. FIGS. 8 and 9 illustrates the viability of JEG-3 extravillous and JAR villous trophoblast-like cell lines, respectively, in PEG hydrogels with varying crosslinkers and RGD adhesion ligand through confocal imaging on days 1, 3 and 7. Cells were stained with Calcein AM (green—live cells) and ethidium homodimer (magenta—dead cells). The cells create cluster structures over time, more so in hydrogels with degradable crosslinkers (VPM, GDQ, and GDQ-W) versus the nondegradable hydrogels (DTT, PEG-DT), where more cell death is also seen. A half-half mix of degradable crosslinkers was also tested that creates bigger structures than fully nondegradable hydrogels.

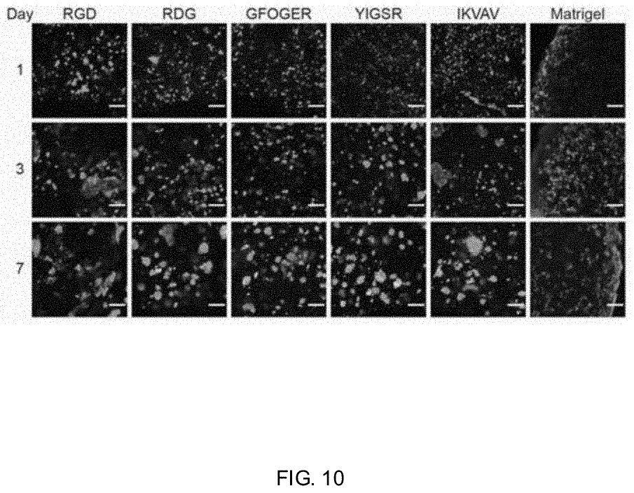

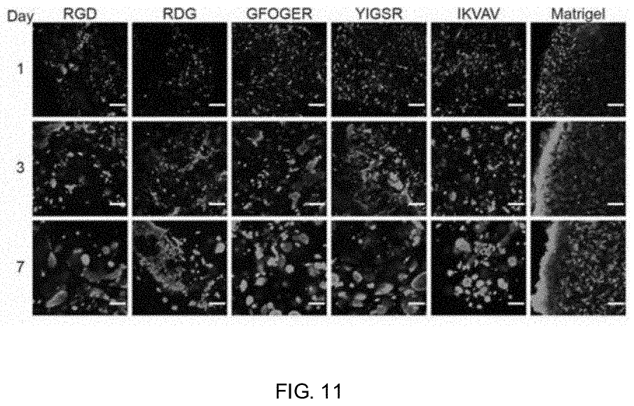

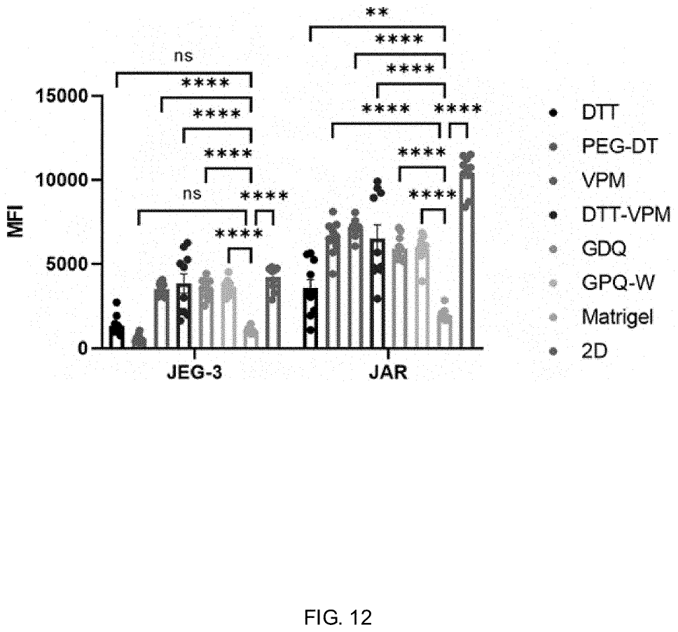

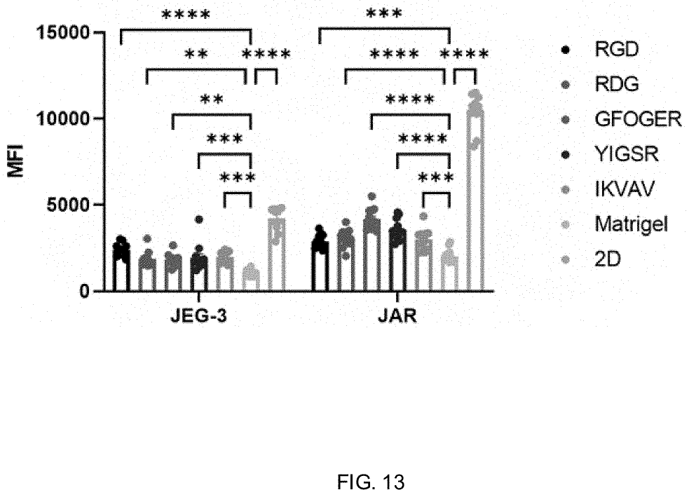

Finding that degradable gels produced the largest trophoblast organoids by day 7, we next investigated the influence of adhesive ligand on cell growth for JEG-3 (FIG. 10) and JAR (FIG. 11) trophoblasts. While choice of crosslinker greatly influenced cell size (FIGS. 8-9), and metabolic activity (FIG. 12), variation in adhesive ligand had little influence on organoid size (FIG. 10-11) or metabolic activity (FIG. 13). Interestingly, metabolic activity and organoid formation was greater in all degradable PEG groups relative to Matrigel controls.

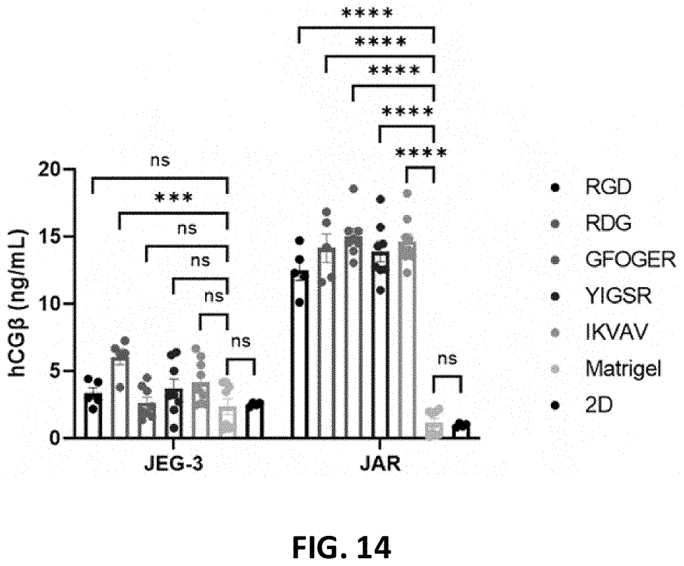

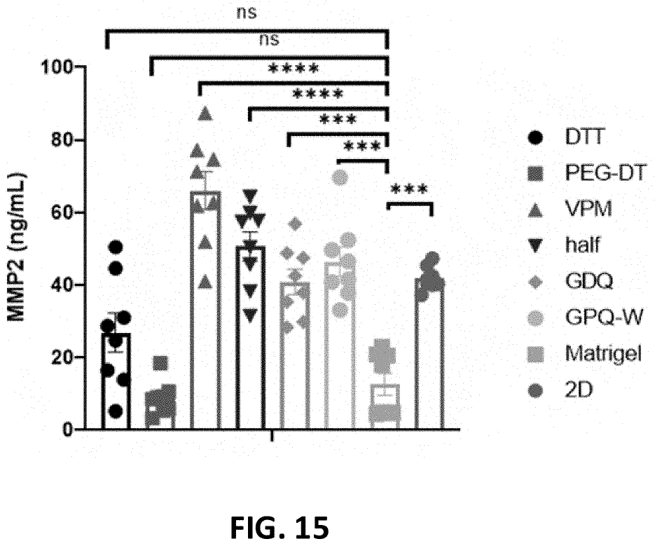

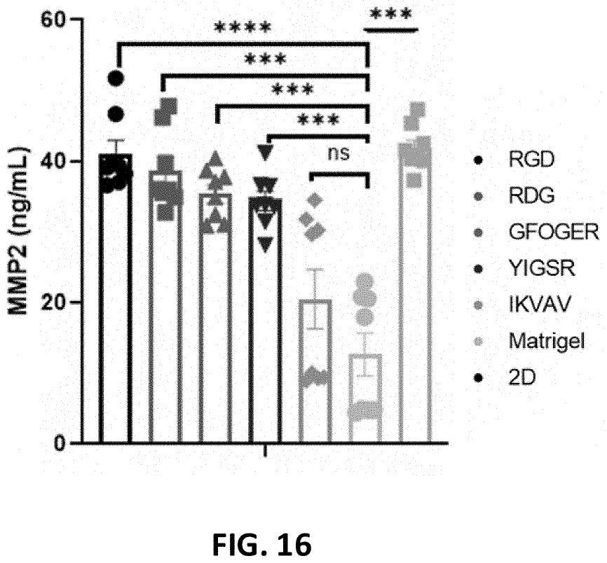

Trophoblast organoids exhibit substantially greater function when grown in three dimensional matrices compared to traditional two dimensional culture. The metabolic activity of cells in 2D culture is two and three times greater on day 7 than JEG-3 and JAR cells grown in degradable 3D matrices, respectively (FIG. 13). However, markers of trophoblast function, such as HCG-beta, exhibit significantly greater secretion in organoid cultures (FIG. 14), particularly in JAR cells. Similarly, MMP-2 secretion in JAR cells is upregulated in degradable matrices (FIG. 15) and regardless of adhesive ligand (FIG. 16).

Collectively, these results show that both EVT- and VT-like trophoblast behavior can be influenced and controlled by synthetic hydrogel matrices to generate immunomodulatory trophoblast-containing constructs.

### REFERENCES

The following references are hereby incorporated by reference in their entirety.
1. Robinson, K A, et al., American Journal of Transplantation, 2018. 18(8): p. 1843-1856.
2. Page, E K, et al., Frontiers in Immunology, 2012. 3: p. 198.
3. Weaver, J D, et al., Tissue engineering Part A, 2015. 21(15-16): p. 2250-2261.
4. Weaver, J D, et al., Acta biomaterialia, 2015. 16: p. 136-144.
5. Headen, D M, et al., Nat Mater, 2018. 17(8): p. 732-739.
6. Franquesa, M, et al., Current opinion in organ transplantation, 2012. 17(4): p. 355-361.
7. Morelli, A E, et al., Nature Reviews Immunology, 2007. 7(8): p. 610.
8. Lu, C Y, et al., Transplantation, 1989. 48(5): p. 848-855.
9. Johnson, K L, et al., Arthritis Rheumatism, 2001. 44(8): p. 1848-1854.
10. Smith, R, et al., Journal of Experimental Medicine, 1977. 146(3): p. 899-904.
11. Weaver, J D, et al., Engineering Artificial Niches for Regenerative Medicine., in Encyclopedia of Tissue Engineering and Regenerative Medicine, R. L. Reis and M. E. Gomes, Editors. 2019, Academic Press: Elsevier. p. 103-108.
12. Weaver, J D, et al., Science Advances, 2017. 3(6): p. e1700184.
13. Weaver, J D, et al., American Journal of Transplantation, 2018.
14. Weaver, J D, et al., Biomaterials, 2018. 172: p. 54-65.
15. Turco, M Y, et al., Nature, 2018. 564(7735): p. 263.
16. Flynn, L, et al., Journal of Biomedical Materials Research Part A, 2006. 79(2): p. 359-369.
17. Huppertz, B, Journal of Clinical Pathology, 2008. 61(12): p. 1296-1302.
18. Atay, S, et al., J Experimental cell research, 2011. 317(8): p. 1192-1202.
19. Tuuli, M, et al., Placenta, 2011. 32: p. S109-S118.
20. Ricordi, C, et al., Nature Reviews Immunology, 2004. 4(4): p. 259-268.
21. Clayton, H, et al., Acta diabetologica, 1993. 30(4): p. 181-189.
22. Beck, J, et al., Tissue engineering, 2007. 13(3): p. 589-599.
23. de Vos, P, et al., Biomaterials, 2006. 27(32): p. 5603-5617.
24. Tuch, B E, et al., Diabetes Care, 2009. 32(10): p. 1887-1889.
25. Jacobs-Tulleneers-Thevissen, D, et al., Diabetologia, 2013. 56(7): p. 1605-1614.
26. Juang, J-H, et al., Transplantation, 1996. 61(11): p. 1557-1561.
27. Pileggi, A, et al., Transplantation, 2006. 81(9): p. 1318-1324.
28. Wang, W, et al., Transplantation, 2003. 76(2): p. 290-296.
29. Suzuki, K, et al., Transplantation, 1998. 66(1): p. 21-28.
30. Hulka, J F, et al., Science, 1968. 161(3842): p. 696-698.
31. Adams, A, et al., Biology of Reproduction, 2001. 64(3): p. 753-763.
32. de Mestre, A M, et al., Reproduction, 2011. 141(6): p. 849-856.
33. Brosnahan, M M, et al., Biology of Reproduction, 2016. 95(6): p. 135, 1-12.
34. Borland, R, et al., Nature, 1970. 228(5271): p. 572.
35. Simmons, R L, et al., American journal of obstetrics, 1967. 97(2): p. 218-230.
36. Tostanoski, L H, et al., Discovery Medicine, 2016. 21(117): p. 403-410.
37. Trowsdale, J, et al., Nature Immunology, 2006. 7(3): p. 241.
38. Chen, Y, et al., Biochemical biophysical research communications, 2013. 436(4): p. 677-684.
39. Kontos, S, et al., Current Opinion in Immunology, 2015. 35: p. 80-88.
40. Hunter, Z, et al., ACS nano, 2014. 8(3): p. 2148-2160.
41. Hess, K L, et al., Adv Funct Mater, 2017. 27(22).
42. Tostanoski, L H, et al., Cell Rep, 2016. 16(11): p. 2940-2952.
43. Cruz-Acuña, R, et al., Nature Cell Biology, 2017. 19(11): p. 1326.
44. Swartzlander, M D, et al., Biomaterials, 2015.41: p. 79-88.
45. Jiang, K, et al., Biomaterials, 2017. 114: p. 71-81.
46. Song, Y, et al., Die Pharmazie—An International Journal of Pharmaceutical Sciences, 2012. 67(5): p. 394-399.
47. Anderson, C S, et al., Macroporous bioengineered scaffolds for cell transplantation. 2011, Google Patents.

1.-142. (canceled)

143. A placenta-mimicking device, comprising:
a. allogeneic cells;
b. trophoblasts; and
c. one or more macroencapsulation devices encapsulating the allogeneic cells, the trophoblasts, or the allogeneic cells and the trophoblasts.

144. The placenta-mimicking device of claim 143, wherein the allogeneic cells are therapeutic cells.

145. The placenta-mimicking device of claim 144, wherein the allogeneic cells are islets, islet cells, or cells capable of producing insulin.

146. The placenta-mimicking device of claim 143, wherein the trophoblasts are primary cells isolated from placenta selected from cytotrophoblasts (CT), syncytiotrophoblasts (ST), extravillous trophoblasts (EVT), or any combination thereof.

147. The placenta-mimicking device of claim 143, wherein the trophoblasts are human trophoblast-like cell lines selected from JEG3, JAR, or any combinations thereof.

148. The placenta-mimicking device of claim 143, wherein the trophoblasts are organoids comprising trophoblasts.

149. The placenta-mimicking device of claim 148, wherein the organoids are prepared by a method comprising:
a. providing or having provided trophoblasts;
b. embedding the trophoblasts in a degradable hydrogel matrix comprising a 3D matrix and one or more adhesive ligands; and
c. incubating the trophoblasts under conditions and for a time sufficient for the trophoblasts to generate the organoids.

150. The placenta-mimicking device of claim 149, wherein the hydrogel matrix comprises poly(ethylene glycol) (PEG), agarose, alginate, or any combination thereof.

151. The placenta-mimicking device of claim 143, wherein the placenta-mimicking device comprises a first macroencapsulation device encapsulating the trophoblasts and a second macroencapsulation device encapsulating the allogeneic cells, wherein a combination of the first macroencapsulation device and the second macroencapsulation device is operable to generate a vaccine when implanted into a subject in proximity to each other.

152. The placenta-mimicking device of claim 143, wherein the macroencapsulation device comprises vascular endothelial growth factor (VEGF).

153. The placenta-mimicking device of claim 143, wherein the macroencapsulation device comprises a nondegradable biocompatible hydrogel matrix.

154. The placenta-mimicking device of claim 149, wherein the hydrogel matrix comprises maleimide-functionalized, 4-arm PEG crosslinked using a nondegradable linker or a 20 kDa 4-arm PEG hydrogel matrix crosslinked using a nondegradable linker.

155. The placenta-mimicking device of claim 154, wherein the nondegradable crosslinkers comprise protease-degradable peptides selected from DTT, PEG-DT, or any combinations thereof.

156. The placenta-mimicking device of claim 149, wherein the hydrogel matrix is functionalized with one or more adhesive peptides, selected from collagen I, collagen IV, laminin, fibronectin, fibrinogen, osteopontin, plasminogen, vitronectin, arginylglycylaspartic acid (RGD), or any combinations thereof.

157. A method of preventing and/or treating an immune disease in a subject in need thereof, the method comprising implanting in the subject a placenta-mimicking device, wherein the placenta-mimicking device comprises:
a. allogeneic cells;
b. trophoblasts; and
c. one or more macroencapsulation devices encapsulating the allogeneic cells, the trophoblasts, or the allogeneic cells and the trophoblasts.

158. The method of claim 157, wherein the allogenic cells comprise donor cells and the immune disease is the subject's immune response to the donor cells.

159. The method of claim 157, wherein the allogenic cells comprise cells capable of producing insulin and the immune disease is diabetes Type I.

160. The method of claim 159, wherein the allogenic cells capable of producing insulin produce insulin in a glucose-dependent manner.

161. The method of claim 157, wherein the placenta-mimicking device comprises a first macroencapsulation device encapsulating the trophoblasts and a second macroencapsulation device encapsulating the allogenic cells, wherein the method further comprises implanting the first macroencapsulation device and the second macroencapsulation device.

162. The method of claim 161 wherein the first encapsulation device is implanted between about 1-30 days prior to implanting the second encapsulation device; and wherein the first and the second encapsulation devices are implanted in proximity to each other.

163. The method of claim 157, wherein the device is implanted in the omentum or subcutaneously.

164. The method of claim 158, wherein the method comprises transplanting donor tissue into the subject concurrently with or prior to implanting the placenta-mimicking device into the subject.
